(19) United States
(12) Patent Application Publication
(10) Pub. No.: US 2023/0147948 A1
(43) Pub. Date: May 11, 2023

(54) SYSTEM COMPRISING PARTICLES AND A REMOVABLE DEVICE

(71) Applicant: XPHELYUM, PARIS (FR)

(72) Inventor: AGNÈS POTTIER, PARIS (FR)

(21) Appl. No.: 17/919,774

(22) PCT Filed: Mar. 23, 2021
(86) PCT No.: PCT/EP2021/057500

§ 371 Date: Oct. 19, 2022

(30) Foreign Application Priority Data
Apr. 20, 2020 (EP) 20315197.2

(51) Int. Cl. A61N 1/36

(52) U.S. Cl. CPC A61N 1/36057; A61N 1/3606

(57) ABSTRACT

The present invention relates to advantageous particles and to a system comprising such particles as well as a removable device, wherein the particles are preferably below 100 μm, are stably interacting with hair follicles, biological cells of the dermis and/or epidermis, LTMRs and/or end-organs, and are preferably activated by a signal emitted by the removable device.

### CROSS-REFERENCE TO RELATED APPLICATION

This application is the U.S. national stage application of International Patent Application No. PCT/EP2021/057500, filed Mar. 23, 2021.

The present invention relates to advantageous particles and to a system comprising such particles as well as a removable device, wherein the particles are preferably below 100 μm, are stably interacting with hair follicles, biological cells of the dermis and/or epidermis, Low Threshold Mechanoreceptors (LTMRs) and/or end-organs, and are preferably activated by a signal emitted by the removable device. These products can be used for sensory enhancement or for creating new sensory means in a subject, in particular a means allowing the perception for example of physical, chemical and/or biological signals which are not perceived by a sense of the subject.

### BACKGROUND

Human brain contains about 86 billion neurons and about 100 trillion synaptic connections forming networks with a set of nodes and connections (i.e., a complex set of relationships or circuits).

Neurons from our peripheral nervous system receive and convey signals (i.e., information), and neurons from our central nervous system process (i.e., neural coding) these signals, the processed signals being at the origin of our perception of the world. Our brain is trained to perceive the world via the stimulation of our five natural senses, namely touch, sight, hearing (and balance), smell and taste. In each neural circuit, neurons convey and process information (i.e., neural coding) using electrical signals (also identified as “electrical impulses”, “electrical spikes” or “action potentials”), whatever the receptors responsible for transmitting the information, i.e., the mechanoreceptors, thermoreceptors or pain receptors involved in touch, the photoreceptors involved in sight, the mechanoreceptors involved in hearing or balance, and the chemoreceptors involved in smell or taste.

This processed information (i.e., neural coding) is dynamically represented by patterns of action potentials generated by neurons in relevant brain regions corresponding to moment-to-moment perceptions, memories, creative thoughts and behaviors.

A first type of proposed neural coding is referred to as the “rate code” model. This model considers that information about the stimulus is encoded by the firing rate of the neurons. In practice, the rate is measured by averaging the number of spikes per second, or a defined (often smaller) time bin before and after stimulus presentation and typically over multiple stimulus trials. This averaging procedure inherently assumes that spike variability reflects noises, and most, if not all, information is conveyed by spike numbers.

A second proposed type of neural coding model is referred to as the “temporal code” model. This model utilizes timing information of spike's discharges to identify the stimulus.

A third proposed type of neural coding model is the “neural self-information” model which postulates that neuronal variability carries itself information. In other words, under this “self-information code model”, any given Inter-Spike-Interval (ISI) is self-tagged with a discrete amount of information [Meng Li et al. Neural Code—Neural Self-information Theory on How Cell-Assembly Code Rises from Spike Time and Neuronal Variability. Frontiers in Cellular Neuroscience, 2017; Volume 11; Article 236].

Understanding/decoding the neural coding (i.e., how information is processed) is still an area of investigation.

Brain Machine Interface (BMI) (also identified as Brain Computer Interface (BCI)) can be defined as a direct communication pathway, through any artificial means, that allows the brain to exchange information directly with an external device. BMI may help understanding how the brain encodes sensory information from the outside world into an internal language, how it integrates external and internal information to produce cognitive/emotional representations and how it generates and executes motor programs [Karen A. Moxon et al. Brain-Machine Interfaces beyond Neuroprosthetics. Neuron 86, Apr. 8, 2015; 54-67]. BMI is seen as a promising means to not only understand but also to achieve neural coding.

In this context, Neuralink has developed ultra-thin multi-electrode polymer probes which are to be inserted in a mammalian brain, offering the possibility of recording neural activity in real time to decode neural information and also the possibility of modulating neural activity to encode new neural information.

However, direct access to the brain may represent a risk to the subject. In addition, with more than about 86 billion of neurons in a human brain, the possibility to get access to even a fraction of neurons remains low.

Moving away from the central nervous system, the peripheral nervous system appears as an interesting alternative for neural coding and peripheral nerve procedures are associated with less risk to the subject.

Sensory restoration devices have so far used the peripheral nervous system to transmit typically sound information (cochlear implants) or image information (optical implants) via implanted (micro)electrodes arrays that send signals (i.e., electrical signals) directly to the appropriate nerve, auditory nerve or optical nerve respectively.

Sensory substitution devices have used the peripheral nervous system too, to stimulate the receptors of intact natural senses. Eyes have the highest capacity for conveying information followed by the ear and the skin. The ear has the highest temporal resolution. So far, it has been possible to “see through the ear or skin” or to “hear through the eyes or skin” [Meike Scheller et al. Chapter 15, Perception and Interactive Technology. Stevens' Handbook of Experimental Psychology and Cognitive Neuroscience, Fourth Edition, edited by John T. Wixted. Copyright 2018 John Wiley & Sons, Inc]. Typically, Braille reading represents a low-tech vision substitution means allowing a subject to “read with the skin”. More technological advanced systems have since emerged. The Tactile Visual Substitution System (TVSS) has been introduced, to convert a tactile stimulation of the skin into visual information. It uses an array of 400 tiny tactile stimulators that transmit information (on the back of the subject) captured by a video camera. Another system using tactile to visual information conversion is the BrainPort device. It uses electro-tactile impulses to stimulate receptors on the surface of the tongue via a flexible electrode array receiving input from a head-mounted video camera. Auditory systems provide a higher spatial acuity and ability for parallel processing and have been developed “to see through the ears”. The system called “vOICe” converts visual images from a camera into sounds by transforming each pixel into a sound. Also, a device able to “hear with the skin” has been developed for deaf people. This device, called Versatile Extra-Sensory Transducer (VEST) from NeoSensory Inc., consists of an array of small vibration motors integrated into a vest. Attached to the vest is a microphone that captures sounds from the environment. These sounds are translated into tactile sensations perceived by the subject via the vibration motors. Finally, somatosensory information that can be usually sensed by the skin, such as temperature, pressure and force, can be captured by sensors and transformed into visual or hearing cues to a subject. However, when using sensory substitution devices, sensory overload is to be avoided. As well, interferences with natural environmental senses are to be avoided.

Low-threshold mechanoreceptors (LTMRs) are special mechanosensitive primary sensory neurons that react to innocuous mechanical stimulation (touch sensation). These cutaneous sensory neurons may be classified as either Aβ, Aδ or C based on their cell body sizes, axon diameter, degree of myelination and axonal conduction velocities. In addition, their firing pattern to sustain mechanical stimuli is variable, ranging from slow (SA) to intermediate (IA) and to rapidly adapting (RA).

LTMRs associated cutaneous end-organs encode touch stimuli and this encoding is then integrated and processed within the central nervous system. Both hairy and hairless (also named non-hairy or glabrous) skin areas contain discrete sets of LTMRs and associated end-organs (also named endings), and these different sets of LTMRs detect specific tactile modalities (Table 1 and FIG. 1). In glabrous skin, four types of LTMRs with fast conduction velocity (Aβ LTMRs) have been defined, each with a distinct terminal morphology (“endings”) and tuning property: (i) Aβ SAI-LTMRs (also herein identified as SAI-LTMRs) innervate Merkel cells in the basal epidermis, (ii) Aβ SA2-LTMRs (also herein identified as SAII-LTMRs) are hypothesized to terminate in Ruffini corpuscles in the dermis, (iii) Aβ RA1-LTMRs (also herein identified as RAI-LTMRs) innervate Meissner's corpuscles in dermal papillae, and (iv) Aβ RA2-LTMRs (also herein identified as RAII-LTMRs) terminate in Pacinian corpuscles deep in the dermis.

In hairy skin, apart from the SA1-LTMR/Merkel cell complex (touch dome), hair follicles are innervated by LTMR termination collars located just below the level of sebaceous gland. Despite their differences in sensitivity and encoding′ ability, the 3 types of lanceolate-ending LTMRs have identical terminal structures [A. Zimmerman et al. The gentle touch receptors of mammalian skin. Science, 2014; 346(6212), 940-954].

The below table 1 identifies LTMRs of the skin and their corresponding end-organs (from A. Zimmerman et al. The gentle touch receptors of mammalian skin. Science, 2014; 346(6212), 940-954; V. E. Abraira et al. The sensory neurons of touch. Neuron (2013); 79(4), 10.1016).

TABLE 1

| Low-threshold | | | | | |
|---|---|---|---|---|---|
| mechanoreceptor | | | | | |
| (LTMR) category | | | | | |
| (encoding non- | Associated fiber | | | | |
| painful stimuli, or | (conduction | | Skin type | End-organ/ | |
| touch stimuli) | velocity) | | innervation | ending type | Location |
| Slowly adapting | Aβ | (16-96 m/s) | Glabrous | Merkel cell | Basal layer of |
| type I: SAI-LTMR | | | | | epidermis |
| | | | Hairy | Merkel cell | Around guard |
| | | | | (touch dome) | hair follicles |
| Slowly adapting | Aβ | (20-100 m/s) | Glabrous | Ruffini endings | Dermis |
| type II: SAII-LTMR | | | Hairy | | |
| Rapidly adapting | Aβ | (26-91 m/s) | Glabrous | Meissner | Dermal papillae |
| type I: RAI-LTMR | | | | corpuscle | |
| | | | Hairy | Longitudinal | Guard/Awl- |
| | | | | lanceolate ending | Auchene hair |
| | | | | | follicles |
| Rapidly adapting | Aβ | (30-90 m/s) | Glabrous | Pacinian | Deep dermis |
| type II: RAII-LTMR | | | | corpuscle | |
| Aδ-LTMR | Aδ | (5-30 m/s) | Hairy | Longitudinal | Awl-Auchene/ |
| | | | | lanceolate ending | Zigzag hair |
| | | | | | follicles |
| C-LTMR | C | (0.2-2 m/s) | Hairy | Longitudinal | Awl-Auchene/ |
| | | | | lanceolate ending | Zigzag hair |
| | | | | | follicles |

Signal Transduction

The conventional view is that primary afferents (i.e., also named primary sensory neurons or LTMRs) are the first to participate to the signal transduction cascade. These neurons have a unique pseudo-unipolar morphology, with a single process that bifurcates into two branches: a distal branch, which can be up to a meter long, that innervates peripheral tissues and a shorter branch that terminates centrally. For the trigeminal system, which covers the head and face, the somata (or cell bodies) of peripheral sensory neurons reside within the trigeminal ganglia and terminate in the medulla. For the rest of the body, the somata reside within the dorsal root ganglia (DRG) and terminate in the spinal dorsal horn or dorsal column nuclei in the medulla. In order for primary sensory neurons to respond to mechanical stimuli and initiate action potentials, they need specific molecular transducers that can be directly activated by physical energy [F. Moehring et al. Uncovering the Cells and Circuits of Touch in Normal and Pathological Settings. Neuron 100, Oct. 24, 2018].

Non-neuronal cells in the periphery were found to contribute intimately with primary sensory neurons to relaying touch signals centrally. Numerous specialized non-neuronal end-organs in the skin sense different features of mechanical stimuli: (1) Merkel cells respond to sustained touch and pressure and aid in two-point discrimination; (2) Ruffini's end-organs sense stretching of skin around objects and over joints; (3) Pacinian corpuscles sense fast vibrations and deep pressure; (4) Meissner's corpuscles sense slow vibrations and changes in texture; and (5) hair follicles detect hair movement in response to very light touch, clothing and air currents. How touch is tuned at each neurite complex is likely due to a variety of factors including the distinct expression patterns and density of mechanotransduction channels in sensory endings that innervate the end-organ and/or the nature of the non-neuronal cells that comprise the structural ending. The response also depends on other factors such as the depth of the end-organs in the skin, the extent of terminal branching, and the types of surrounding non-neuronal cells [F. Moehring et al. Uncovering the Cells and Circuits of Touch in Normal and Pathological Settings. Neuron 100, Oct. 24, 2018].

Sensory restoration or sensory substitution devices use the Peripheral Nervous System (PNS), by either directly stimulating the appropriate nerve fibers in the context of sensory restoration, typically using electrodes (with or without wireless connection) or by stimulating the receptors/nerves of different senses in the context of sensory substitution (for example mechanoreceptors of the skin or ears, photoreceptors of the eyes, chemoreceptors of the nose or tongue). However, directly stimulating the nerves using electrodes requires the stability of the interactions between the electrode(s) and neurons with time and an appropriate selectivity of the electrode allowing a relevant electrode/neurons interaction [Hannes P. Saal et al. Biomimetic approaches to bionic touch through a peripheral nerve interface. Neuropsychologia 79 (2015) 344-353]. As such, electrodes—by design (due to their size, hardness, composition, etc.)—may not be the optimized tools for stimulating neurons.

US2011/0071439 describes stimulators implanted in the skin to deliver a tactile stimulation, wherein the stimulators are magnetic particles (preferably sized 2 mm or less) fixed in an array. When an input signal is applied to a transmitter, it is transformed into a signal causing the motion of a corresponding stimulator.

Also, while the stimulation of receptors of our senses is interesting for sensory substitution, these receptors are usually engaged during sensory substitution and are not available for other tasks (see for instance Braille to “see/read with its skin/fingertips”).

Therefore, there is a need for new tools allowing efficient, ideally improved, neural coding (i.e., processing of information), to the benefice of people requiring sensory restoration or substitution.

### SUMMARY OF THE INVENTION

Inventor herein provides a minimally (or non) invasive and highly efficient tool/system (i.e., with appropriate spatial and temporal resolutions) spectacularly improving neural coding. The herein described system creates spatiotemporal electrical patterns at the level of the peripheral nervous system which can be efficiently read by the brain and are able to restore a perception, for example touch perception, in a subject who is deprived of it, to substitute a perception means to another in a subject suffering of an altered perception (for example of an altered vision or an altered hearing), to enhance perception in a subject, and/or to create new perception means in a subject. The system of the invention also enables for the first time the brain of a subject, for example of a human subject, to perceive beyond the reality the subject is used to perceiving thanks to his senses.

Herein described is a system (A) comprising particles (B) and a removable device (C). Preferred particles (B) are (sized)/have a size below 100 μm, are stably interacting with hairs, hair follicles, biological cells of the dermis and/or epidermis, Low Threshold Mechanoreceptors (LTMRs) and/or end-organs, preferably with hair follicles, biological cells of the dermis and/or epidermis, Low Threshold Mechanoreceptors (LTMRs) and/or end-organs, and are activable by a signal emitted by the removable device (C). The removable device (C) typically collects an input signal which is, optionally processed and, used to activate the particles (B), the removable device being wearable by a subject.

In a preferred aspect, the size of particles (B) is below 100 μm, the particles (B) are stably interacting with hair follicles, biological cells of the dermis and/or epidermis, Low Threshold Mechanoreceptors (LTMRs) and/or end-organs of a subject, the particles (B) are prepared from a material selected from a conductor, a semiconductor, a semiconductor with direct bandgap, an insulator, a piezoelectric and a magnetoelectric material, and are activable by a signal emitted by the removable device (C). The removable device (C) collects an input signal which is, optionally processed and, used to activate the particles (B), the removable device being wearable by a subject.

In a particular aspect, the particles are prepared from a material selected from a conductor, a semiconductor, a semiconductor with direct bandgap, a piezoelectric and a magnetoelectric material.

In another particular aspect, the particles are prepared from a material selected from a conductor, a semiconductor, a semiconductor with direct bandgap, an insulator and a magnetoelectric material.

In a further particular aspect, the particles are prepared from a material selected from a conductor, a semiconductor, a semiconductor with direct bandgap and an insulator material.

In again another particular aspect, the particles are prepared from a material selected from a conductor, a semiconductor and a semiconductor with direct bandgap.

This system is, according to a particular aspect of the invention, used for sensory enhancement in a subject, or for creating new sensory means in a subject, said new sensory means allowing the perception of a physical signal, chemical signal and/or biological signal which is not perceived by a sense of the subject, for example by a human sense.

A particular herein described system (A) is a sensory restoration system, a sensory substitution system, a sensory enhancement system, or a new sensory perception system.

Also herein described according to another particular aspect of the invention, are particles (also herein identified as “particles (B)”) for use for touch sensory restoration in an amputee or in a burn victim, or for sensory substitution in a subject at least partially or totally deprived of taste, smell, hearing, balance and/or vision, when particles interact with hairs, hair follicles, biological cells of the dermis and/or epidermis, LTMRs and/or end-organs, preferably with hair follicles, biological cells of the dermis and/or epidermis, Low Threshold Mechanoreceptors (LTMRs) and/or end-organs of the subject, and when particles are activated by an external source of energy, wherein particles are below 100 μm (i.e., each particle is a below 100 μm-particle), and are prepared from a material selected from a conductor, a semiconductor, a semiconductor with direct bandgap, an insulator, a piezoelectric and a magnetoelectric material, in particular from a conductor, a semiconductor, a semiconductor with direct bandgap, a piezoelectric and a magnetoelectric material.

Further herein described is a composition for use for touch sensory restoration in an amputee or in a burn victim, or for sensory substitution in a subject at least partially, or totally, deprived of taste, smell, hearing, balance and/or vision, wherein the composition comprises particles (also herein identified as “particles (B)”), and wherein said particles are below 100 μm, are prepared from a material selected from a conductor, a semiconductor, a semiconductor with direct bandgap, an insulator, a piezoelectric and a magnetoelectric material, and are activated by an external source of energy, in particular from a conductor, a semiconductor, a semiconductor with direct bandgap, a piezoelectric and a magnetoelectric material.

In a preferred aspect, the composition is a liquid, in particular a tattoo ink, or a gel.

In another preferred aspect, the composition comprising the particles or the particles themselves is/are part of a needle, in particular of a microneedle, for example of the tip of a needle or of a microneedle.

The present description also encompasses any kit comprising at least two of the herein described products, for example at least one or two distinct populations of particles (B), preferably together with a removable device (C), and optionally together with a tool (such as one or more needles, one or more microneedles, a patch, an injector, etc.) designed to appropriately deposit and/or position the particles (B) at the adequate site of the subject's body.

Inventors herein describe a kit comprising at least two distinct populations of particles, optionally together with a tool designed to deposit and/or position particles at the adequate site of the subject's body for them to stably interact with hair follicles, biological cells of the dermis and/or epidermis, Low Threshold Mechanoreceptors (LTMRs) and/or end-organs of a subject, wherein the size of particles is below 100 μm, and particles are prepared from a material selected from a conductor, a semiconductor, a semiconductor with direct bandgap, an insulator, a piezoelectric and a magnetoelectric material, and are activable. They also describe a kit comprising particles (B), a removable device (C), and one or several tools selected from a sensor such as an electrode, a memory and a processor, wherein the removable device (C) is wearable by a subject, the size of particles (B) is below 100 μm, and particles (B) are i) prepared from a material selected from a conductor, a semiconductor, a semiconductor with direct bandgap, an insulator, a piezoelectric and a magnetoelectric material, and ii) activable by a signal emitted by the removable device (C).

### DETAILED DESCRIPTION OF THE INVENTION

Inventor herein advantageously describes a system (A) comprising particles (B) and a removable device (C), wherein particles (B) are below 100 μm, are stably interacting with hairs, hair follicles, biological cells of the dermis and/or epidermis, Low Threshold Mechanoreceptors (LTMRs) and/or end-organs, preferably with hair follicles, biological cells of the dermis and/or epidermis, Low Threshold Mechanoreceptors (LTMRs) and/or end-organs, and are activable by a signal emitted by the removable device (C), and wherein the removable device (C) collects an input signal which is, optionally processed and, used to activate the particles (B), the removable device being preferably wearable by a subject.

A preferred herein described system (A) comprises particles (B) and a removable device (C), wherein the size of particles (B) is below 100 μm, the particles (B) are stably interacting with hair follicles, biological cells of the dermis and/or epidermis, Low Threshold Mechanoreceptors (LTMRs) and/or end-organs of a subject, the particles (B) are prepared from a material selected from a conductor, a semiconductor, a semiconductor with direct bandgap, an insulator, a piezoelectric and a magnetoelectric material, in particular from a conductor, a semiconductor, a semiconductor with direct bandgap, a piezoelectric and a magnetoelectric material, and are activable by a signal emitted by the removable device (C). The removable device (C) collects an input signal which is, optionally processed and, used to activate the particles (B), the removable device being wearable by a subject.

In a particular aspect, the particles (B) are prepared from a material selected from a conductor, a semiconductor, a semiconductor with direct bandgap, a piezoelectric and a magnetoelectric material.

In another particular aspect, the particles (B) are prepared from a material selected from a conductor, a semiconductor, a semiconductor with direct bandgap, an insulator and a magnetoelectric material.

In a further particular aspect, the particles (B) are prepared from a material selected from a conductor, a semiconductor, a semiconductor with direct bandgap and an insulator material.

In again another particular aspect, the particles are prepared from a material selected from a conductor, a semiconductor and a semiconductor with direct bandgap.

In the context of the present invention, the subject is a subject having a brain, typically an animal, in particular a mammal, preferably a human, whatever its age or sex and health status.

A particular subject is a subject suffering of an altered perception, i.e., a subject suffering of an altered perception related to the lack of functioning or to the malfunctioning of one or more of his senses, typically a mammalian subject who doesn't see, hear, smell, taste, touch and/or balance, or who doesn't see, hear, smell, taste, touch and/or balance correctly, for example a diseased subject (a patient).

In such subjects, the present invention is typically used for “sensory restoration”, i.e., to restore a subject's body particular functionality or full functionalities of the subject's body, or for “sensory substitution”, thereby allowing the substitution of a particular functionality of the subject's body or full functionalities of the subject's body.

Another subject is a healthy subject who wants to experience sensory enhancement (“feel more/feel better”), i.e., who wants to better perceive outside stimuli (still within the natural possibilities offered by his/her senses), or a subject who wants to experience new perception, i.e., who wants to perceive a reality beyond the reality accessible through senses.

The biological cells particles (B) are to interact with are preferably selected from a keratinocyte, melanocyte, Merkel cell, Langerhans cell, fibroblast, mast cell, macrophage, lymphocyte and platelet. The LTMRs particles (B) are to interact with are preferably selected from SAI-LTMR, SAII-LTMR, RAI-LTMR, RAII-LTMR, Aδ-LTMR and C-LTMR.

The end-organs particles (B) are to interact with are preferably selected from Ruffini corpuscle, Meissner corpuscle, Pacinian corpuscle and longitudinal lanceolate ending.

In a particular aspect of the invention, particles (B) interact with hairs, hair follicles, biological cells of the dermis and/or epidermis, LTMRs and/or end-organs, preferably with hair follicles, biological cells of the dermis and/or epidermis, Low Threshold Mechanoreceptors (LTMRs) and/or end-organs, for example with biological cells of the dermis and/or epidermis, with LTMRs and with end-organs.

### Particles Composition: Temporal and Spatial Resolutions

The particles of the invention (“particles (B)”) are intended to work through an “on”/“off” mode of action, meaning that when they are activated by an external means, typically by an external source of energy, preferably an external manmade source of energy, as further described herein below, the particles of the invention act as transducers and convert an incoming signal (i.e., typically the signal emitted by the removable device (C)) into an output signal of different nature, or modulate/relay locally an incoming signal (i.e., typically the signal emitted by the removable device (C)), thereby acting on peripheral nerves to convey an information to the brain for neural coding (i.e., processing of information). In other words, if not stimulated, the particles do not transmit any signal.

In particular, the material the particles are made of does not conserve an overall net magnetization (contrary to what is observed for example with ferrimagnetic particles) which would be detrimental to the efficient technical effect allowed by the invention (i.e., the coding effect).

The material constituting the particles of the invention as well as their structure are key to obtain the desired efficacy. Indeed, this efficacy directly depends on the efficiency of the conversion of an input energy into an output energy (input energy signal transduction), or on the efficiency of the modulation and conversion of an input energy into an output energy (input energy signal modulation). A careful selection of the composition and structure (i.e., an amorphous structure, a semi-crystalline structure or a crystalline structure) of the particles therefore optimizes the temporal energy input signal transduction and/or the temporal energy input signal modulation.

The conversion of an input energy into an output energy (input energy signal transduction), or the modulation of an input energy into an output energy (input energy signal modulation) is triggered when the particles of the invention are stimulated by/activated by/exposed to the input energy. In the context of the present invention “activated by”, “stimulated by” and “exposed to” can be used interchangeably. The expression “exposed to” is more specifically used when insulating particles as herein described below are considered.

### Conductor Particle

The particle of the invention can be a conductor particle with an electrical bulk conductivity σ of at least 1×10⁴ S/m at 20° C., preferably of at least 1×10⁵ S/m at 20° C., for example of at least 1×10⁶ S/m at 20° C., typically of at least 1×10⁷ S/m at 20° C., the electrical bulk conductivity corresponding to the electrical conductivity of the bulk material. A preferred conductor particle can be selected from a metal particle, a crystallized metal oxide particle, an amorphous oxide particle, a transition metal dichalcogenide particle, a particle made with carbon atoms, an organic particle, and any mixture thereof.

When the particle is a metal particle, it is typically made of gold (Au) element (“gold particle”), Copper (Cu) element (“copper particle”), Molybdenum (Mo) element (“molybdenum particle”), Aluminum (Al) element (“aluminum particle”), Palladium (Pd) element (“palladium particle”), platinum (Pt) element (“platinum particle”), or any mixture thereof. Preferably, it is made of gold (Au) element, platinum (Pt) element or a mixture thereof.

When the particle is a crystallized metal oxide particle, it typically comprises rhenium element. The particle can typically be a rhenium (VI) trioxide (ReO₃) particle or a rhenium (IV) dioxide particles (ReO₂, also named rhenium oxide particle).

When the particle is an amorphous oxide particle, it typically consists of a mixture of at least two metal elements, typically indium and tin to form the indium-tin oxide (ITO) particle, indium and zinc to form the indium-zinc oxide (IZO) particle, or aluminum and zinc to form the aluminum-zinc oxide (AZO) particle.

When the particle is a transition metal dichalcogenide particle, it is typically the FeS₂ particle, the FeSe₂ particle, the FeTe₂ particle, the TaS₂ particle, the TaSe₂ particle, the TaTe₂ particle or the NbSe₂ particle.

When the particle is a particle made with carbon atoms (i.e., a carbon-based particle), it has typically a graphene structure, a single-wall carbon nanotube structure, a multi-wall carbon nanotube structure, a reduced graphene oxide structure, a graphite structure, a carbon black structure.

When the particle is an organic particle, it is typically made of polypyrrole, polyaniline, polythiophene or a derivative thereof such as Poly(3,4-ethylenedioxythiophene) or Poly(3,4-ethylenedioxythiophene)-poly(styrenesulfonate).

Also herein described are particles comprising a mixture of any one of the herein above described conductor materials as well as particles having a core-shell structure, the core and the shell being prepared from distinct conductor materials, each material being selected from any one of the herein above described conductor materials, and their uses in the context of the present invention.

These conductor particles are (directly or indirectly) in contact with the peripheral nervous system at hairs, hair follicles, biological cells of the dermis and/or epidermis, LTMRs and/or end-organs location, preferably at hair follicles, biological cells of the dermis and/or epidermis, LTMRs and/or end-organs location, and, when activated by an external electrical source of energy, modulate, for example stimulate, or simply relay, locally, the electrical signal to the peripheral nerves. As further explained in details below, this local modulation/relay action is made thanks to an output signal readable by a stimulator module (c2) comprising an electrical source of energy used to activate the particles as selected in the context of the invention.

Alternatively, these conductor particles, when made with carbon atoms, for example graphene particles, are typically activated by an external light source of energy and come into contact with the peripheral nervous system to stimulate the peripheral nerves. The signal's transduction is made thanks to an output signal readable by a stimulator module (c2) which comprises a light source of energy used to activate the particles as selected in the context of the invention, the light signal being converted by the particles into an electrical signal.

### Semi-Conductor Particle

The particle of the invention can be a semi-conductor particle with an electrical bulk conductivity σ of at least 1×10⁻³ S/m at 20° C., preferably between 1×10⁻³ S/m and 1×10² S/m at 20° C., even more preferably below 1×10² S/m at 20° C., even more preferably of at least 1×10⁻² S/m at 20° C., between 1×10⁻² S/m and 1×10² S/m at 20° C., or below 1×10² S/m at 20° C., the electrical bulk conductivity corresponding to the electrical conductivity of the bulk material.

A preferred semi-conductor particle can be selected from a metal oxide particle, an organic particle, a particle made with silicon or germanium atoms, a transition metal dichalcogenide particle, a quantum dot, a perovskite particle, and any mixture thereof.

When the particle is a metal oxide particle, it typically consists of a mixture of at least two metal elements, typically of three metal elements such as indium, gallium and zinc to form an indium-gallium-zinc oxide (a-IGZO) particle. The metal oxide particle may also be prepared with a single metal element, typically the zinc element to form a zinc oxide (ZnO) particle, the titanium element to form titanium dioxide (also named titanium oxide) (TiO₂) particle, the tin element to form tin oxide (SnO) particle.

When the particle is an organic particle, it typically consists in, or comprises, small molecules or polymers, for example pentacene, poly(3-hexylthiophene) (P3HT), poly(diketopyrrolopyrrole-terthiophene) (PDPP3T), 5,50-bis-(7-dodecyl-9H-fluoren-2-yl)-2,20-bithiophene (DDFTTF) and/or polyisoindigobithiophene-siloxane (PiI2T-Si).

When the particle is made of silicon, it typically has an amorphous (a-Si) structure, a polycrystalline structure or a crystalline structure.

When the particle is made of germanium, it typically has an amorphous structure or a crystalline structure.

When the particle is a transition metal dichalcogenide particle, it is typically a MoS₂ particle, a MoSe₂ particle, a MoTe₂ particle, a WS₂ particle, a WSe₂ particle, a ReS₂ particle, a ReSe₂ particle, a FeSe particle or a HfS₂ particle.

When the particle is a quantum dot particle, it is typically a GaN quantum dot, a InN quantum dot, a SnO quantum dot, a ZnO quantum dot, a ZnS quantum dot, a SnS quantum dot, a SnSe quantum dot, a FeSe quantum dot, a CdS quantum dot, a CdSe quantum dot, a ZnSe quantum dot, a CdTe quantum dot, a ZnTe quantum dot, a InSb quantum dot, a GeSe quantum dot, a InAs quantum dot, a GaAs quantum dot, a InP quantum dot, a GeTe quantum dot, a GaSb quantum dot, a Germanium quantum dot, a Silicon quantum dot, a graphene quantum dot, a SnTe quantum dot, a ternary I-III-VI₂ quantum dot where I is typically the copper (Cu) element or the Silver (Ag) element, III is typically the Aluminum (Al) element, the gallium (Ga) element, the indium (In) element or the bismuth (Bi) element, and VI is typically the sulfur (S) element, the selenium (Se) element or the tellurium (Te) element. Ternary quantum dots are typically CuInSe₂, AgBiTe₂ or AgBiSe₂.

When the particle is a perovskite particle, it has typically the following structures ABX₃, ABCX₃, or ABCDX₆ (corresponding to a double perovskite structure), where A is an organic or an inorganic element, B, C and D are inorganic elements, and X is an halide ion or oxygen. Typically, the particle is KBaTeBiO₆ or Ba₂AgIO₆.

Also herein described are particles comprising a mixture of any one of the herein above described semi-conductor materials as well as particles having a core-shell structure, the core and the shell being prepared from distinct semi-conductor materials, each material being selected from any one of the herein above described semi-conductor materials, and their uses in the context of the present invention, for example in a method as herein taught.

These semi-conductor particles are (directly or indirectly) in contact with the peripheral nervous system at hairs, hair follicles, biological cells of the dermis and/or epidermis, LTMRs and/or end-organs location, preferably at hair follicles, biological cells of the dermis and/or epidermis, LTMRs and/or end-organs location, and, when activated by an external electrical source of energy, modulate, for example stimulate, or simply relay, locally, the electrical signal to the peripheral nerves. As further explained in details below, this local modulation/relay action is made thanks to an output signal readable by a stimulator module (c2) comprising an electrical source of energy used to activate the particles as selected in the context of the invention.

Alternatively, these semi-conductor particles, when possessing a direct band gap, are typically activated by an external light source of energy and come into contact with the peripheral nervous system to stimulate the peripheral nerves. The signal's transduction is made thanks to an output signal readable by a stimulator module (c2) which comprises a light source of energy used to activate the particles as selected in the context of the invention, the light signal being converted by the particles into an electrical signal (i.e., photoelectric conversion).

### Insulator Particle

The particle of the invention can be an insulator particle with an energy gap of at least 4 eV, the energy gap corresponding to the separation between the valence band and the conduction band. A preferred insulator particle can be selected from a metal oxide particle, a mixed metal oxide particle, boron nitride (BN) particle, an organic particle, and any mixture thereof.

When the particle is a metal oxide particle, the particle can typically be an yttrium oxide particle (Y₂O₃), a tantalum pentoxide particle (Ta₂O₅), a hafnium dioxide particle (HfO₂, also named hafnium oxide particle) or a zirconium dioxide particle (ZrO₂, also named zirconium oxide particle).

When the particle is a mixed metal oxide particle, it typically consists of a mixture of at least two metal elements and oxygen, typically silicon (Si), aluminum (Al) and oxygen to form an aluminosilicate particle.

When the particle is an organic particle, it is typically made of an organic polymer or co-polymer such as an acrylate polymer or co-polymer, a polyurethane, a polycarbonate, or a polytetrafluoroethylene. When the insulating biocompatible organic material is made of an acrylate polymer or co-polymer, it is typically prepared from acrylate monomers such as ethyl acrylate monomers, ethylene-methyl acrylate monomers, methyl methacrylate monomers, 2-chloroethyl vinyl ether monomers, 2-hydroxyethyl acrylate monomers, hydroxyethyl methacrylate monomers, etc. A typical polymer particle can be the polymethylmethacrylate (PMMA) particle or the poly(2-hydroxyethyl methacrylate) particle.

These insulator particles are (directly or indirectly) in contact with the peripheral nervous system at hairs, hair follicles, biological cells of the dermis and/or epidermis, LTMRs and/or end-organs location, preferably at hair follicles, biological cells of the dermis and/or epidermis, LTMRs and/or end-organs location, and, when activated by an external electrical source of energy, modulate, for example inhibit, or simply relay, locally, the electrical signal to the peripheral nerves. As further explained in details below, this local modulation/relay action is made thanks to an output signal readable by a stimulator module (c2) comprising an electrical source of energy used to activate the particles as selected in the context of the invention.

### Piezoelectric Particle

The particle of the invention can be a piezoelectric particle, typically a piezoelectric particle having a structure and/or composition capable of converting an external mechanical input signal into an internal electrical output signal.

When the particle is a piezoelectric particle, it typically consists of a quartz (SiO₂) particle, a barium titanate (BaTiO₃) particle, a AlN particle, a GaN particle, a ZnO particle, a boron nitride (BN) particle or a particle comprising or consisting in polyvinylidene fluoride polymer or derivative thereof, polymeric L-lactic acid, polymeric D-lactic acid, DNA or M13 bacteriophage.

These piezoelectric particles are (directly or indirectly) in contact with the peripheral nervous system at hairs, hair follicles, biological cells of the dermis and/or epidermis, LTMRs and/or end-organs location, preferably at hair follicles, biological cells of the dermis and/or epidermis, LTMRs and/or end-organs location, and, when activated by an external mechanical source of energy, stimulate, locally, the peripheral nerves. As further explained in details below, this stimulation is made thanks to an output signal readable by a stimulator module (c2) which comprises a mechanical source of energy used to activate the particles as selected in the context of the invention, the mechanical signal being converted by the particles into an electrical signal (i.e., piezoelectric conversion).

Alternatively, these piezoelectric particles may be activated by an external electrical source of energy and once (directly or indirectly) in contact with the peripheral nervous system, stimulate, locally, the peripheral nerves. This stimulation is made thanks to an output signal readable by a stimulator module (c2) which comprises an electrical source of energy used to activate the particles as selected in the context of the invention, the electrical signal being converted by the particles into a mechanical signal (i.e., reverse piezoelectric conversion).

### Magnetoelectric Particle

The particle of the invention can be a magnetoelectric particle. When the particle is a magnetoelectric particle, it typically consists in a composite particle having a core consisting in a material exhibiting a spinel structure such as CuFe₂O₄ or CoFe₂O₄, and a shell consisting in a material exhibiting a perovskite structure such as BaTiO₃.

These magnetoelectric particles are (directly or indirectly) in contact with the peripheral nervous system at hairs, hair follicles, biological cells of the dermis and/or epidermis, LTMRs and/or end-organs location, preferably at hair follicles, biological cells of the dermis and/or epidermis, LTMRs and/or end-organs location, and, when activated by an external magnetic source of energy, stimulate, locally, the peripheral nerves. As further explained in details below, this stimulation is made thanks to an output signal readable by a stimulator module (c2) which comprises a magnetic source of energy used to activate the particles as selected in the context of the invention, the magnetic signal being converted by the particles into an electrical signal (i.e., magnetoelectric conversion).

The below table 2 summarizes the different options offered by the particles selected in the context of the invention, depending on their specific composition and structure (typically an amorphous structure, a semi-crystalline structure or a crystalline structure), said composition and structure dictating the particles' ability to locally convert (i.e., transduce) or modulate/relay an incoming output signal, readable by a stimulator module (c2), into an electric signal or into a mechanic signal, preferably into an electric signal, which will stimulate the peripheral nerves, thereby allowing sensory restoration, sensory substitution, sensory enhancement or new sensory perception.

In a particular aspect, particles of different compositions and structures can be used simultaneously. A typical kit herein described comprises at least two of the herein described products, for example at least two distinct populations of particles (B). The populations of particles of such a kit can typically be administered in vivo in the same biological spot or on different biological spots. Alternatively, the particles of different compositions and structures can be mixed (physically), or connected directly (i.e., in physical contact, for example in a core/shell disposition) or indirectly (i.e., via a linker) before being administered in vivo.

TABLE 2

| transducer and/or | | | |
|---|---|---|---|
| energy modulator | | | |
| particle composition | | | |
| at temperature | Input | Output | Conversion |
| typically between | external | internal | mode of |
| 15° C. and 45° C. | energy | energy | energy |
| Conductor particle | Electric | Electric | — |
| such as Au particle | | | |
| or ReO₂ particle | | | |
| Semiconductor | Electric | Electric | — |
| particle such as Si | | | |
| particle or FeSe | | | |
| particle | | | |
| Semiconductor | Light | Electric | Photoelectric |
| quantum dot such as | | | |
| Si quantum dot | | | |
| (possessing a direct | | | |
| band gap) | | | |
| Insulator particle | Electric | Electric | — |
| such as BN particle | | | |
| Piezoelectric | Mechanic | Electric | Piezoelectric |
| particle such as | | | |
| BaTiO₃ particle | | | |
| or BN particle | | | |
| Piezoelectric | Electric | Mechanic | Reverse |
| particle such as | | | Piezoelectric |
| BaTiO₃ particle | | | |
| or BN particle | | | |
| Magnetoelectric | Magnetic | Electric | Magnetoelectric |
| particle such as | | | |
| CoFe₂O₄/BaTiO₃ | | | |
| particle | | | |

In order to deliver efficient interaction with the peripheral nerves (i.e., achieving high spatial resolution), the selected particles of the invention advantageously stably interact with hairs, hair follicles, biological cells of the dermis and/or epidermis, LTMRs and/or end-organs, preferably stably interact with hair follicles, biological cells of the dermis and/or epidermis, LTMRs and/or end-organs.

The expression “stably interacting” indicates that particles (i) do not significantly move once administered (typically injected), i.e., at least, preferably more than 20%, 30%, 40%, 50%, 60%, 70%, 80% or 90%, even more preferably more than 80% or 90%, of the injected particles remain at the site of injection, the particles interacting either directly with hair follicles, biological cells of the dermis and/or epidermis, LTMRs and/or end-organs, or indirectly with hair follicles, biological cells of the dermis and/or epidermis, LTMRs and/or end-organs, i.e., the particles interact with elements of the biological medium present at hair follicles, biological cells of the dermis and/or epidermis, LTMRs and/or end-organs locations, and/or with the biological cells (i.e., in particular with the membranes of the biological cells and/or are up-taken by the biological cells), and (ii) do not degrade after injection/administration unless expressly designed to experience in vivo degradation (for example via dissolution of the particle). By stably interacting with elements of the biological medium, the particles ensure a high spatial resolution for signal transmission.

Typically, at the site of injection:

the biological cells of interest comprise keratinocytes, melanocytes, Merkel cells, Langerhans cells, fibroblasts, mast cells, macrophages, lymphocytes, and/or platelets; and

the biological medium of interest comprises:

Low Threshold Mechanoreceptors (LTMRs), the LTMR being a slowly adapting type I (SAI-LTMR), a slowly adapting type II (SAII-LTMR), a rapidly adapting type I (RAI-LTMR), a rapidly adapting type II (RAII-LTMR), a Aδ-LTMR or a C-LTMR, and

End-organs, the end-organs being typically Meissner corpuscles, Ruffini corpuscles, Pacinian corpuscles and/or longitudinal lanceolate endings.

To allow the required “stable interaction” state between the particles and the elements of the biological medium, for example the biological cells of the dermis and/or epidermis, LTMRs and/or end-organs, the two following particle's features are to be properly selected: (i) the size of the particles and (ii) the composition of the particles' core and/or of the particles' surface coating, in order to optimize the particle's “design”.

### (i) The Size of the Particles

In the context of the present invention, the particles' size is typically below about 100 μm. A threshold limit of the particles' size of about 200 nm has been observed regarding uptake and retrograde transport of particles following axonal delivery in cortical neuronal cells [Anna Lesniak et al. Rapid growth cone uptake and dynein-mediated axonal retrograde transport of negatively charged nanoparticles in neurons is dependent on size and cell type. Small, 2018, 1803758]. Uptake and retrograde transport were observed for particles having a size up to 100 nm whereas they were hardly observed for particles having a size typically above about 200 nm. In the context of the present invention wherein the stable interaction between particles and elements of the biological medium including biological cells is required, the size of the particles is preferably comprised between about 200 nm and about 100 μm.

The size of the particles is typically measured using well-known electron microscopic (EM) tools or light scattering tools.

When the particles are hydrophobic or hydrophilic and the size of the particles is at the microscale, i.e., between about 1 μm and about 100 μm, the size is typically measured using an electron microscopic technic, typically scanning electron microscopy. The longest dimension of the core of a particle (the core of the particle being the particle without any surface coating) measured in the electron microscopy image is reported. At least 100 particles of a population are measured in their longest dimension and the median size of the considered population of particles is calculated. In this context, the “size” of the particles designates the median size of the particles of a population comprising at least 100 particles.

When the particles are hydrophilic and the size of the particles is at the nanoscale, i.e., between about 1 nm and about 1000 nm, and when the particle size is monodisperse, i.e., the polydispersity index of the suspension of particle is found typically below 0.2, the size is typically measured using the Dynamic Light Scattering (DLS) technique. In the context of the DLS technic, the size of the particle is typically measured when the particles are in aqueous suspension (i) at a pH between about 6.5 and about 7.5, (ii) at a particles' concentration between 0.5 g/kg and 10 g/kg (weight/weight), the particles concentration being typically measured by dry extract (i.e., the suspension containing the particles is typically placed at a temperature between 100° C. and 250° C. for a duration period typically comprised between 15 minutes and overnight) or by ICP-MS or by ICP-OES and (iii) at an ionic strength presenting an electrical conductivity typically comprised between about 0.01 μS/cm and about 2000 μS/cm at a temperature between about 15° C. and about 25° C. When measured by DLS, the size of the particles corresponds to the size of the particles given in intensity.

Alternatively, the electron microscopy technique (EM), typically the transmission electron microscopy (TEM) or Cryo-TEM can be used to measure the size of the particles at the nanoscale. In this case, the longest dimension of a particle measured in the electron microscopy image is reported. At least 100 particles are measured in their longest dimension and a median size of the particles of the population comprising at least 100 particles is calculated. In this context, the “size” of the particles designates the median size of the particles of a population comprising at least 100 particles.

When the particle size is polydisperse (i.e., the polydispersity index of the suspension of particle is found typically above 0.2 when measured by DLS), a fractionation technique can be used to separate the populations of particles in different monodisperse particles' size populations. Typically, a field flow fractionation tool can be used to reach this goal. The size of the particles in each population/fraction is determined as described herein-above using DLS or EM tools. In addition, in each population/fraction the quantity of particles is estimated. This estimation can typically be done by quantification of at least one element constituting the particle. The “size” of the particles represents in this context the average weight of the sizes of the particles obtained in each population of particles or fraction thereof.

The shape of particle (B) is not critical for the invention. Typically, the particle can have an “inhomogeneous shape”. The expression “inhomogeneous shape” designates particle the sizes of which have been measured in the 3 dimensions (x, y, z) and present one or two dimensions larger than the other(s), typically one or two dimensions more than about three times larger than the other(s). However, a particle with a homogeneous shape is preferred. The expression “homogeneous shape” designates particle the sizes of which have been measured in the 3 dimensions (x, y, z) and present a ratio which does not exceed a factor 3 between each dimension (i.e., x/y≤3, y/z≤3 and z/x≤3).

Whatever the surface of the particles, they will most certainly end up within biological cells when their size is below about 20 μm or 10 μm. On the contrary, whatever the surface of the particles, they will be localized preferentially outside biological cells, typically in the biological medium surrounding the cells when their size is typically above about 10 μm or 20 μm.

Indeed, when the size of the particles is typically below 20 μm, the particles can enter cells by different mechanisms: through endocytosis-dependent pathways or direct cytoplasmic delivery [Nathan D. Donahue, et al. Concepts of nanoparticle cellular uptake, intracellular trafficking, and kinetics in nanomedicine. Advanced Drug Delivery Reviews 143 (2019) 68-96]. Endocytic-dependent pathways encompass five mechanistically distinct classes: (a) clathrin-dependent endocytosis for particles' size typically between about 100 nm and about 500 nm; (b) caveolin-dependent endocytosis for particles' size typically between about 50 nm and about 100 nm; (c) clathrin- and caveolin-independent endocytosis; (d) phagocytosis, typically used by immune cells, including macrophages, dendritic cells, neutrophils, and B lymphocytes, for particles' size typically up to about 20 μm, preferentially up to about 10 μm; and (e) micropinocytosis for particles' size typically between about 0.5 μm and about 1.5 μm [Nathan D. Donahue, et al. Concepts of nanoparticle cellular uptake, intracellular trafficking, and kinetics in nanomedicine. Advanced Drug Delivery Reviews 143 (2019) 68-96].

Alternatively, direct injection of particles into the cytoplasm of biological cells may be performed in the context of the present invention, the biological cells being preferably selected from keratinocytes, melanocytes, Merkel cells, Langerhans cells, fibroblasts, mast cells, macrophages, lymphocytes, platelets and any combination thereof. For this, several technics may be used via biochemical or physical means, including electroporation or microinjection [Nathan D. Donahue, et al. Concepts of nanoparticle cellular uptake, intracellular trafficking, and kinetics in nanomedicine. Advanced Drug Delivery Reviews 143 (2019) 68-96].

In any case, the size of the particles below about 100 μm will ensure relevant (i.e., stable) interaction between the particles of the invention and the biological medium where the particles have been injected. A direct interaction of the particle with the cell is possible, and the particle can even operate from within the cell. The particles having the same size-scale as a cell, they are capable of significantly enhancing smooth interactions both with the biological cells and with the biological medium.

(ii) The Composition of the Particles and/or of the Particles' Surface Coating Agent

The composition of the core of the particles as presented herein above is typically selected according to the source of energy provided by the device (C). However, when the composition of the particles' core presents potential safety issues for the subject (i.e., a potential dissolution process triggering direct release of potentially toxic metal elements in the biological medium, and/or a potential redox phenomenon at the surface of the particle triggering oxidation of proteins or biological moieties or generation of reactive oxygen species (ROS), and/or a potential catalytic property of the particles), a surface coating is preferably applied using a surface coating agent.

This surface coating is preferably an inorganic surface coating limiting, ideally preventing, any potential degradation of the organic surface coating agent (such as bond breaking) upon time, ex-vivo (i.e., upon storage of the particles prior use) or in vivo (i.e., upon injection of the particles in vivo). Indeed, the surface coating agent should ideally not contain carbon-carbon bonds or any bonds susceptible of being broken ex vivo or in vivo (typically due to oxidation phenomenon). Also, when an organic coating agent is selected, precaution regarding sterilization and storage of the particles are to be taken to prevent potential degradation of the surface coating agent, typically due to oxidation reactions. In any case, the surface coating agent should be selected so that potential residues or moieties typically resulting from oxidation reactions and/or bond breaking of the surface coating agent do not triggered in vivo toxicity.

Also, when a surface coating agent is applied to the particles in a particular aspect of the description, this surface coating agent should not desorb from the particles' core. Therefore, it is key to consider coating agent able to establish strong bonds/links with the surface of the particles, typically able to establish complexing or covalent bonds. Typical covalent bonds are found between silane-based compounds (i.e., coating agents) and the surface of oxide particles. Other very strong bonds (i.e., bonds considered as exhibiting a strength intermediate between the strength exhibited by covalent bonds and that exhibited by complexing bonds) are found between phosphate-based or phosphonate-based compounds (i.e., coating agents) and the surface of oxide particles, or between thiol-based compounds and the surface of metal particles, quantum dots or semiconductor particles.

In any configuration (i.e., whatever the selection of the core of the particle and/or of the surface coating agent), the particles should be stable (i.e., physically and chemically stable) prior their injection in a subject (unless specified that the particles should degrade in vivo). When the particles are in suspension (i.e., dispersed in solution), typically prior injection, they should form a stable suspension. A suspension is considered as stable in the absence of observed sedimentation of the particles (i.e., the appearance of the suspension is homogeneous) within typically 1 minute, 2 minutes, or 3 minutes following manual agitation of the suspension. Also, when the particles are in suspension, the supernatant solution collected by any means and free of particles, should present no or a very low amount of elements constituting the particles and/or constituting the particles' surface coating (i.e., the detected amount of elements should be in the limit of resolution of the technic selected for quantifying these elements which are well-known by the skilled person in the art such as the ICP-MS (inductively coupled plasma—mass spectrometry) or the ICP-OES (inductively coupled plasma—optical emission spectrometry) technics).

The surface of the particles is typically hydrophilic or hydrophobic. An hydrophilic surface ensures the wettability of the particles in aqueous medium and the possibility to obtain a water suspension. Alternatively, an hydrophobic surface is not wet by water. However, a particle with an hydrophobic surface can be wet by biological entities (such as proteins absorption on the surface of the hydrophobic particles), present in the biological medium. Particles contact angles with water measurement represents a typical wettability measurement to assess particles' hydrophobicity. In such measurement, particles are dispersed in ultrapure water and left to dry on a substrate to create a homogeneous layer of particles. The contact angle measurement is performed with water as probe liquid, at room temperature. Typical hydrophobic particles have a contact angle with water above about 50°, preferably above about 60°, 70°, 80° or 90°. Typical hydrophilic particles have a contact angle with water below about 30°, preferably below about 25°, 20° or 10°. Quantification of nanoparticle surface's hydrophobicity is also proposed by comparing nanomaterial binding affinity to two or more engineered collector [Andrea Valsesia et al. Direct quantification of nanoparticle surface hydrophobicity. Communications Chemistry, 2018, 1:53].

When the particles are hydrophilic and have a size typically below about 20 μm, preferably below about 10 μm, they have typically a surface charge below about +30 mV to avoid any potential in vivo toxicity triggered by the surface charge. In this context, the surface charge is typically measured through the so-called zeta potential of the particles, the particles being in a water solution (i) at a pH between about 6.5 and about 7.5, (ii) at a particles' concentration between 0.5 g/kg and 10 g/kg (weight/weight), the particles concentration being typically measured by dry extract (i.e., the suspension containing the particles is typically placed at a temperature between 100° C. and 250° C. for a period typically comprised between 15 minutes and overnight) or by ICP-MS or ICP-OES and (iii) at an ionic strength presenting an electrical conductivity typically between about 0.01 μS/cm and about 2000 μS/cm at a temperature between about 15° C. and about 25° C.

### Particles' Formulation and Compositions

Particles (B) are typically formulated in a liquid, in particular in a tattoo ink, or in a gel. In a particular example, the particles can be formulated in a liquid that turns into a gel when administered to a subject.

When the transition from a liquid to a gel is triggered by a change of temperature, the liquid-to-gel transition typically occurs between 30° C. and 40° C. Poly(D,L-lactic acid-co-glycolic acid)-b-poly(ethylene glycol)-b-poly(D,L-lactic acid-co-glycolic acid) (PLGA-PEG-PLGA) triblock copolymers typically are materials which exhibit a sol-gel transition upon heating. The liquid-to-gel transition temperature is typically affected by the following parameters: the concentration of copolymer, the chain length of PEG, the chain length of PLGA, the molar ratio between PEG and PLGA, or the lactic acid/glycolic acid (LA:GA) ratio within the PLGA. All these parameters can be easily adjusted by the skilled person to trigger a liquid-to-gel transition at a temperature typically comprised between 30° C. and 40° C., for example at the human body temperature.

The herein defined particles are typically part of a composition which is a liquid or a gel, in particular a liquid having a liquid-to-gel transition temperature between 30° C. and 40° C.

When using a liquid that turns into a gel when administered to a subject, a controlled release of the particles at the site of administration can be obtained by an adaptation of the gel according to methods well-known by the skilled person in the art. Depending on the affinity between the particles and the gel, a controlled release of the particles, typically between few seconds (for example about two seconds) and 1 week, can be obtained. Alternatively, according to the kinetic of degradation of the gel, a controlled release of the particles, typically between 1 hour and 1 week, can be obtained.

The affinity between the particles and the gel is typically characterized by the type of bonding existing between the particles and the material constituting the gel. The bonding can typically be a hydrogen bonding, a bonding resulting from electrostatic interactions, a complexing bonding or a chemically cleavable covalent bonding.

The degradation of the gel typically consists in the swelling (i.e., expansion) of the gel or the breaking of bonds in the material(s) constituting the gel. The gel is ideally biodegradable. A biodegradable gel can typically comprises hydrolytic degradable polyesters blocks, such as poly(ε-caprolactone) (PCL) blocks and poly(D,L-lactide-co-glycolide) (PLGA), blocks. Alternatively, the biodegradable gel can comprise polymer blocks with enzymatically degradable peptides, such as poly(L-alanine) (PA) blocks and chitosan blocks.

The particles (B) or composition comprising such particles can be directly administered using typically a syringe and a needle when particles are in suspension (i.e., when they are formulated as a liquid or as a gel, provided the viscosity of the gel remains compatible with such administration mode, for example as a liquid that turns into a gel when administered in a subject). When formulated as a gel, the particles can also be deposited on the surface of the skin. Particles will penetrate in the dermis and epidermis for example spontaneously or by massage. In this particular cases, hydrophobic particles are preferred.

Alternatively, the particles can be directly stuck to the surface of a needle and the particles are released in the biological medium typically between few seconds (typically at least two seconds) and 10 minutes following needle insertion into the skin typically up to the dermis layer. Also, the particles can be formulated as a gel which stuck to the surface of a needle, the gel being released in the biological medium typically between few seconds and 10 minutes upon needle insertion in vivo. To stick the particles or the gel containing the particles to the surface of the needle while allowing the rapid release of the particles or of the gel containing the particles from the needle when in vivo, a linker agent containing a chemical cleavable bond or a UV cleavable bond can typically be used. This linker agent binds the particle or the gel containing the particles to the surface of the needle. The linker agent is typically a linker agent containing a chemical cleavable bond such as a cleavable disulphide bond, a cleavable ester bond, or a cleavable hydrazone bond.

The particles can become the principal component of the needle(s), microneedle(s), or of the tip(s) of the needle(s) or microneedle(s). In such case, the needle(s), microneedle(s), or the tip(s) of the needle(s) or microneedle(s) is(are) inserted in vivo and remain(s) there. The erosion (such as degradation or dissolution) of the needle(s) or microneedle(s), or of the tip(s) of the needle(s) or microneedle(s), triggers the release of the particles, typically within seconds (for example about 2 seconds), hours or days following needle(s) or microneedle(s) insertion/implantation. The needle(s) or microneedle(s) are left in the skin for a selected period and can be removed at any time by extracting the part(s) of the needle(s) or microneedle(s) that has/have not been dissolved. Dissolvable needle(s) or microneedle(s) or dissolvable tip(s) of the needle(s) or microneedle(s) typically comprise(s) water soluble polymers, such as polyvinyl alcohol, polyvinylpyrrolidone or polyvinyl acetate, sugars, or any mixture thereof.

The dissolvable needle(s) or microneedle(s) or tip(s) of needle(s) or microneedle(s) comprise(s) the herein described particles.

In a preferred aspect, needle insertion is that observed in the context of tattoo procedure, i.e., insertion is non-invasive and is not considered as a physical intervention on the human or animal body. Inventors also herein describe a composition for use for touch sensory restoration in an amputee or in a burn victim, or for sensory substitution in a subject at least partially deprived of taste, smell, hearing, balance and/or vision, wherein the composition comprises particles, and wherein particles are below 100 μm, are prepared from a material selected from a conductor, a semiconductor, a semiconductor with direct bandgap, an insulator, a piezoelectric and a magnetoelectric material, for example from a material selected from a conductor, a semiconductor, a semiconductor with direct bandgap, a piezoelectric and a magnetoelectric material, in particular from a conductor, a semiconductor and a semiconductor with direct bandgap, preferably from a conductor, a semiconductor, a semiconductor with direct bandgap, an insulator and a magnetoelectric material, even more preferably from a conductor, a semiconductor, a semiconductor with direct bandgap and an insulator, and are activated by an external source of energy.

As explained herein above, the source of energy may be selected from an electrical source, a light source, a mechanical source and a magnetic source. Typically:

i) when the source of energy is an electrical source, the particle is preferably prepared from a material selected from a conductor, a semi-conductor and a piezoelectric material, more preferably from a conductor, a semi-conductor, an insulator and a piezoelectric material, even more preferably from a conductor, a semi-conductor and an insulator material;
ii) when the source of energy is a light source, the particle is preferably prepared from a material selected from a semiconductor with a direct band gap material and a conductor made of carbon atoms; iii) when the source of energy is a mechanical source, the particle is preferably prepared from a piezoelectric material; and
iv) when the source of energy is a magnetic source, the particle is preferably prepared from a magnetoelectric material.

In a preferred embodiment, the composition is a liquid, in particular a tattoo ink, or a gel, or the composition, or the particles it contains, is/are part of a needle, a microneedle, or of a tip of a needle or microneedle.

In a particular example, the composition is in the form of a liquid that turns into a gel once administered to a subject.

Administration Volume and Concentration of Particles at Hairs, Hair Follicles, Biological Cells of the Dermis and/or Epidermis, LTMRS and/or End-Organs Location

The volume occupied by particles per administration/injection site/area at hairs, hair follicles, biological cells of the dermis and/or epidermis, LTMRs and/or end-organs location, preferably at hair follicles, biological cells of the dermis and/or epidermis, LTMRs and/or end-organs location, is typically between about 0.001 mm³ (i.e., 0.001 μL) and about 100 mm³ (i.e., 100 μL), preferably between about 0.005 mm³, about 0.01 mm³, about 0.05 mm³, about 0.1 mm³, about 0.5 mm³, about 1 mm³, about 2 mm³, or about 5 mm³, and about 10 mm³, about 20 mm³, or about 50 mm³. The volume occupied by particles per administration/injection site/area is for example between about 0.02 mm³ and about 20 mm³. If several administrations/injections are performed in a given (biological) area, the volume described in the present paragraph is that resulting from one administration/injection only. The volume occupied by the particles corresponds to the minimum volume measured in vivo (typically using imaging technics well known by the skilled person) which includes all the administered, typically injected, particles. Because the particles remain at their administration site, the volume occupied by the particle corresponds to the administered volume (e.g., the volume of the administered liquid or gel or the volume of the needle, microneedle or tip of the needle or microneedle which has dissolved).

When multiple administrations/injections of particles per (biological) area/administration spot are performed, the total volume of the particles at the biological area/administration spot corresponds to the sum of the volumes occupied by the particles after each single administration step.

Needle(s) or microneedle(s) which can be used to administer/inject the particles, has(have) typically the following dimensions: a diameter typically between about 0.10 mm, or more than about 0.10 mm, and about 0.50 mm or about 0.40 mm, and a length typically between about 1 mm, about 1.5 mm, about 2 mm, or about 5 mm and about 100 mm or about 50 mm.

The concentration of particles per administration/injection site/area [in a given (biological) area, if several administrations/injections are performed, the concentration of particles described in the present paragraph is that resulting from one administration/injection only] at hairs, hair follicles, biological cells of the dermis and/or epidermis, LTMRs and/or end-organs location, preferably at hair follicles, biological cells of the dermis and/or epidermis, LTMRs and/or end-organs location, is typically between about 1 mg/100 g (weight of particles by weight of biological medium) and about 40 g/100 g, preferably between about 1 g/100 g and about 20 g/100 g. Because the particles remain at their administration site/injection location, the concentration of particles at hairs, hair follicles, biological cells of the dermis and/or epidermis, LTMRs and/or end-organs location, preferably at hair follicles, biological cells of the dermis and/or epidermis, LTMRs and/or end-organs location, corresponds to the concentration of particles which is present in the suspension to be administered in vivo (e.g. the concentration of particles in the liquid or gel or the concentration of particles in the needle, microneedle or tip of the needle or microneedle).

### Stable Interaction Between the Device (C) and Particles (B)

In a preferred aspect of the invention, the removable device (C) advantageously stably interacts (in particular during the activation/stimulation step(s)) with particles.

In another preferred aspect, both the removable device (C) and particles (B) are not located at a biological area of the subject corresponding to fingertips, mouth, lips and foot soles. This is to limit, ideally avoid, interference with critical sensory biological area of human body. In other words, the removable device (C) and particles (B) are localized/present on a biological area which is distinct of fingertips, mouth, lips and foot soles, and the removable device (C) and particles (B) are preferably advantageously stably interacting together (in particular during the activation/stimulation step(s)).

Particles (B) can be administered at multiple biological areas of a subject, typically 2, 3, 4, 5, 6, 7, 8, 9 or 10 different biological areas, which are preferably distinct of mouth, lips, fingertips and foot soles. The surface of a biological area represents typically about 0.5 cm², about 1 cm², about 2 cm², about 3 cm², about 4 cm², about 5 cm², about 6 cm², about 7 cm², about 8 cm², about 9 cm², about 10 cm², about 15 cm², or about 20 cm² of the skin of the subject.

Multiple administrations (targeting multiple administrations/injection spots at one or multiple biological areas) of particles or composition comprising particles, typically more than one administration and up to typically 1000 administrations at one or multiple biological areas (each area comprising one or multiple spots) of the subject are typically performed.

In other words, multiple administrations (at one or several administrations/injection spots) of particles or composition comprising the particles are performed per biological area. For example, 1, 2, 3, 4, 5, 6, 7, 8, 9, 10, 11, 12, 13, 14, 15, 16, 17, 18, 19, 20, 25, 30, 40, 50, 60, 70, 80, 90, 100, 150, 200, 250, 300, 400 or 500 administrations are performed per biological area.

When multiple administrations of particles or composition comprising particles are performed, the distance between two adjacent administrations is typically of less than about 100 or of about 100 μm, about 200 μm, about 300 μm, about 400 μm, about 500 μm, about 600 μm, about 700 μm, about 800 about 900 μm or about 1000 μm.

Typical biological areas where the particles can be valuably administered are for example areas where the subject is used to wear jewelry (such as a ring, a bracelet, a necklace) such as for example the subject's arm, leg or ankle.

In a particular aspect of the invention, particles are to be administered once on a given site of implantation where they will ensure reproducible electrical signal/information transmission. In another particular aspect of the invention, particles are to be administered several times on a given site of implantation. Repeated/successive administrations of particles on a given site can typically be performed in order to increase the number of injection sites in the subject, the already administered particles being still usable when designed as non-biodegradable particles (i.e., used as “permanent” and “re-usable” neural interfaces).

In another particular aspect where transient effect is sought, biodegradable particles may be selected instead.

Thus, the present invention advantageously allows spatiotemporal control of the stimulation of primary afferents (i.e., also identified as primary sensory neurons or LTMRs) through particles' activation.

In a particular aspect of the invention, neural data of a subject, such as data obtained from BOLD signal using functional Magnetic Resonance Imaging (fMRI) or electroencephalography (EEG) signals, may be recorded to assess the efficacy of neural coding in the subject. Neural data can also be recorded at the peripheral nerve system level (typically via a sensor, such as electrode(s)). These neural data can then be used as a feedback loop in order to “train” the system of the invention and/or to “train” the subject him/herself. Indeed, the information transmitted to the brain thanks to the system of the invention can be recorded (on a memory) and then processed by a processor, and then decoded (using the recorded/processed neural data and typically machine learning for neural decoding). The processed data can then advantageously be sent back to the subject in the form a signal perceivable by any one of the five natural senses of the subject in order to accelerate the learning process and facilitate any sensory restoration process, sensory substitution process, sensory enhancement process, or new sensory perception process. In a particular aspect, the decoded information obtained from a given subject can be used by said subject (i.e., transmitted to the subject and perceived by the subject) to facilitate learning and exploitation of information. Alternatively, the decoded information can be sent to the removable device (C) to update and improve output signal transmission. These records can be used as herein above described thanks to the stable interaction existing (under activation) between the removable device (C) and the implanted/injected particles (B).

In an aspect of the invention, such record(s) may be used to lock the system (A) of the invention by specifically associating the removable device (C) of the invention with the particles (B) of the invention.

### Device

In a preferred aspect of the invention, the removable device (C) comprises a collector module (c1) the function of which is to collect an input signal. The input signal is typically selected from a physical signal, a chemical signal and a biological signal, and is used to activate the particles (B). The collector module (c1) is capable of processing the signal when required.

A particular removable device (C) of the invention thus collects an input signal which is, optionally processed and, used to activate the particles (B). The removable device is preferably wearable by a subject.

A typical device (C) of the invention comprises a collector module (c1) collecting an input signal which is typically a physical signal, a chemical signal or a biological signal, and capable of processing the signal when required, and a stimulator module (c2).

In a particular aspect, the collector module (c1) comprises a module (c1′) collecting an input signal and a processing module (c1″) encoding/converting the input signal into an output signal readable by the stimulator module (c2).

In another typical aspect, the stimulator module (c2) comprises a source of energy which is selected from an electrical source, a light source, a magnetic source and a mechanical source, said source using the output signal to activate the particles (B).

As taught herein above, the collector module (c1) collects a signal which is typically a physical signal, a chemical signal or a biological signal or several signals, e.g., physical, chemical and/or biological signals.

A physical signal is for example an electromagnetic signal (see FIG. 2) such as a radio wave signal, a microwave signal, a visible light signal, an infrared signal, an ultraviolet (UV) signal, an X-ray signal, a gamma-ray signal, etc.; a thermal radiation/heat signal; an electric signal; a magnetic signal; or a mechanic signal such as for example an ultrasound signal, a pressure signal or a strain signal.

The collector module can be a sensor module.

It is typically a “physical sensor module”, i.e., a sensor module collecting a physical phenomenon (i.e., a physical signal).

A physical sensor module can be an “image sensor module” detecting information in the form of light. An image sensor module typically consists of integrated circuits that sense the information and convert it into an equivalent current or voltage which can be later converted into digital data.

A physical sensor module can also be an “ultrasonic sensor module”. An ultrasonic sensor module is typically used to measure the distances between the sensor and an obstacle object. The ultrasonic sensor module generally works on the principle of the Doppler Effect and includes an ultrasonic transmitter and a receiver. The ultrasonic transmitter transmits the signal in one direction and this transmitted signal is then reflected back whenever there is an obstacle and is received by the receiver. The total time required for the signal to be transmitted and then received back is generally used to calculate the distance between the ultrasonic sensor and the obstacle.

A physical sensor module or physical sensor can also be for example an “infrared” sensor module; a “tactile” sensor module; a “pressure” sensor module; a “strain” sensor module; a “temperature” sensor module; a “magnetic-based” sensor (magnetometer) module; an “optical” sensor module; an “acoustic-based” sensor module; a “gravity” sensor (accelerometer) module; an “angular rate” sensor (gyroscope) module or a “deep pressure” sensor (barometer) module.

The sensor module (c1) can also be a “chemical sensor module” or “chemical sensor”, i.e., a module collecting a chemical phenomenon (i.e., a chemical signal). When the sensor module is a chemical sensor module, it is typically a liquid or gas sensor module detecting the composition or the concentration of a chemical agent in a medium such as for example an organic molecule or an ion.

The sensor module (c1) can also be a “biological sensor module” or “biosensor”, i.e., a module collecting a biological phenomenon (i.e., a biological signal). When the sensor module is a biosensor, it typically detects the composition or concentration of a biological agent such as for example a protein a nucleic acid a cell, a bacterium or a virus, in a medium.

Each sensor module is capable of processing the signal (if and when required) and typically combines sensing, computation, communications and power means into a very small volume typically below 100 mm³, below 10 mm³, or even below 1 mm³. A sensor module or several sensor modules, typically two or three sensor modules, or even a network of sensor modules can be combined in the device to increase its sensing ability. For instance, an optical sensor module can be coupled with an ultrasound sensor module to increase its sensing ability.

The collector module collecting the input signal can also be any other suitable means capable of collecting an input signal from one or several sensors (physical, chemical and/or biological sensors), or from one or several computing systems, for example any data generated by a computing system and transmitted in the form of a digital electrical signal, the sensor(s) or computing system(s) being external to the device. The input signal received by the collector module can be any input signal sent by remote sensor(s) and/or remote computing system(s), through wired (such as for example a HDMI or USB connector) or wireless connection, preferentially via a wireless connection such as for example Bluetooth and WIFI.

The herein described system is in particular used for sensory enhancement in a subject, or for creating new sensory means in a subject allowing the perception of a physical signal, chemical signal and/or biological signal which are not perceived by a sense of the subject.

The sensory restoration, sensory substitution, sensory enhancement, or new sensory perception system according to the invention, preferably comprises a sufficient number “X” of dimensions or parameters and, in each dimension, a sufficient level “N” of features to build a robust information that will create or re-create sensory perception. Coding signal (corresponding to the signal emitted by the source of energy from the stimulator module) can typically have dimensions expressed for example as intra-signals frequency, inter-signals frequency, signals amplitude, signals intensity, signals waveform, signals repetition, signals repetition frequency, signals total time, and any combination thereof. As well, in each of these dimensions, “N” features can be implemented to (i) reconstitute a well-known perception such as a sound, a melody, colors, hue and luminance of a landscape, distance and direction, etc. and/or to (ii) create a new perception such as for example infrared vision or ultrasound vision. Machine learning can typically be used by the skilled person for such neural coding, or for implementing neuronal network methods to handle, typically transmit and/or record, information.

When the collector module (c1) comprises a module (c1′) collecting an input signal and a processing module (c1″) encoding/converting the input signal into an output signal readable by a stimulator module (c2), the processing module (c1″) preferably contains a deep learning framework determining the parameters required to generate an output signal readable by the stimulator module. Machine learning can typically be used to encode information and implement a neuronal network method or system capable of determining the required parameters. In one aspect, the module (c1′) transmits signals to the processing module (c1″) which uses ADC (Analog-to-Digital Converter) or an equivalent converter, to perform the digitization of (digitalize) the acquired analog signals and generate an output signal which is then sent to a stimulator module (c2). For instance, the signal processing can be an image analysis, a text analysis (i.e., data analysis) or a speech analysis. The signal is captured by a module (c1′) of the collector module and sent to a processing module (c1″), for example for color segmentation, radiance segmentation, hue segmentation, sentence segmentation, or word segmentation. Then, the processing module converts this input signal into an output signal and sends it to the stimulator module (c2).

In another context, a sensor module can sense a change of a measured parameter using a module (c1′) and transfer the information corresponding to this change to a processing module (c1″) (which can typically be a microcontroller) that calculates and converts the change into an output signal (containing all information from the input signal) readable by a stimulator module (c2).

The herein described stimulator module (c2) of the system (A) of the invention comprises a source of energy which is selected from an electrical source, a light source, a mechanical source and a magnetic source, said source using the signal to activate the particles (B).

Each collector module encodes an input signal into an output signal readable by a stimulator module, typically encodes/converts the input signal into an output signal readable for example by a light source of energy, an electric source of energy, a mechanical source of energy, or a magnetic source of energy, preferably by a light source of energy or an electric source of energy. Typically, the output signal can be an electrical signal to be sent to a stimulator module (c2) comprising (micro)electrodes acting as an electrical source of energy to activate the particles (B). Alternatively, the output signal can be an electrical signal to be sent to a stimulator module (c2) comprising (micro)LEDs acting as a light source of energy to activate the particles (B). According to another aspect, the output signal can be an electrical signal to be sent to a stimulator module (c2) comprising (micro)motors acting as a mechanical source of energy to activate the particles (B). In a further distinct aspect, the output signal can be an electrical signal to be sent to a stimulator module (c2) comprising (micro)electromagnets acting as a magnetic source of energy to activate the particles (B).

Several stimulator modules, typically two or three stimulator modules, or a network of stimulator modules, can be combined in the device to increase its sensing ability. Several devices can also be used in parallel to increase sensing ability.

The spikes, generated in response to input signal(s) from the collector module(s), confirm the successful reading of the output signal by the stimulator module(s) present in the system (A) as well as the successful stimulation of the particles by the source of energy and consecutive induced stimulation of the peripheral nerves which will then convey/transmit a signal to the central nervous system which it can interpret (FIG. 3). These spikes can be recorded as electrophysiological signals and observed and/or decoded.

These spikes are generated in response to input signal(s) from the collector module(s), thanks to (i) the stable interaction existing (under activation) between the removable device (C) and the implanted/injected particles (B), and (ii) the stable interaction existing between particles (B) of the invention and elements of the biological medium present at hair follicles, biological cells of the dermis and/or epidermis, LTMRs and/or end-organs locations.

The removable device (C) is preferably powered by an external source or by a battery which is part of the device.

### Wearable Device Design

The wearable device (C) is typically included in a jewelry, in a clothing or in a medical device. When included in a jewelry, it may be included for example in a ring, in a bracelet or in a necklace. When included in a clothing it may be included for example in a tee-shirt, in a sweatshirt, in a sock, in a mitt or in a glove, provided that it delivers reliable external stimulation to the particles administered/implanted under the subject's skin. When included in a medical device, it may be included for example in an artificial skin (for example an ‘electronic skin’), in a patch or in a bandage.

In a particular aspect, the device (C) is a bracelet, a ring, a necklace, an artificial skin, a patch, a bandage, a mitt or a glove.

As herein indicated, the stimulator module (c2) preferably comprises a source of energy which is selected from an electrical source, a light source, a magnetic source and a mechanical source, said source using the output signal to activate the particles (B).

In a particular system of the description:

i) when the source of energy is an electrical source, the particle is prepared from a material selected from a conductor, a semi-conductor and a piezoelectric material, preferably from a conductor, a semi-conductor, an insulator and a piezoelectric material, even more preferably from a conductor, a semi-conductor and an insulator material;
ii) when the source of energy is a light source, the particle is prepared from a material selected from a semiconductor with a direct band gap material and a conductor made of carbon atoms;
iii) when the source of energy is a mechanical source, the particle is prepared from a piezoelectric material;
iv) when the source of energy is a magnetic source, the particle is prepared from a magnetoelectric material.

### Stimulation Parameters

When the source of energy used to activate the particles is an electrical source, the electrical stimulation (i.e., the signal) intensity (i.e., current intensity) is typically between 0.1 μA and 10 mA, the electrical stimulation (i.e., the signal) frequency is typically between 1 Hz and 500 Hz, the electrical stimulation (i.e., the signal) pulse width is typically between 5 μs and 500 ms, and the electrical stimulation (i.e., the signal) waveform is typically a square, a rectangle or a triangle waveform, said square, rectangle or triangle waveform being monophasic, biphasic-charge balanced or biphasic-charge imbalanced, or a pulson (i.e., a square pulse divided in short bursts of square pulses) waveform.

When the source of energy used to activate the particles is a light source, the light stimulation (i.e., the signal) wavelength is typically within the infrared or near infrared (i.e., corresponding to a wavelength typically above 650 nm, preferably equal to or above 800 nm), because of its ability to penetrate deeper into the tissue. The incoming light input source is preferentially selected based on the particle composition to optimize the conversion of the signal emitted by the light source into an electrical signal. In addition, when the energy source to activate the particles is a light source, the light stimulation (i.e., the signal) irradiance rate is typically between 0.1 mW/mm² and 1000 mW/mm², the light stimulation (i.e., the signal) frequency is typically between 1 Hz and 500 Hz, the light stimulation (i.e., the signal) pulse width is typically between 5 μs and 500 ms, and the light stimulation (i.e., the signal) waveform is typically a square, a rectangle or a triangle waveform, or a pulson (i.e., a square pulse divided in short bursts of square pulses) waveform.

When the source of energy used to activate the particles is an external mechanical input source, the mechanical source has typically an amplitude between 0.1 μm and 1000 μm. In addition, when using an external mechanical input source, the mechanical stimulation (i.e., the signal) frequency is typically between 1 Hz and 500 Hz, the mechanical stimulation (i.e., the signal) pulse width is typically between 5 μs and 500 ms, the mechanical stimulation (i.e., the signal) waveform is typically a square, a rectangle, or a triangle waveform, or a pulson (i.e., a square pulse divided in short bursts of square pulses) waveform.

When the source of energy used to activate the particles is an external magnetic input source, the magnetic source has typically a permanent magnetic field between 1 mT and 500 mT. In addition, when using an external mechanical input source, the magnetic stimulation (i.e., the signal) frequency is typically between 1 Hz and 500 Hz, the magnetic stimulation (i.e., the signal) pulse width is typically between 5 μs and 500 ms, the magnetic stimulation (i.e., the signal) waveform is typically a square, a rectangle, or a triangle waveform, or a pulson (i.e., a square pulse divided in short bursts of square pulses) waveform.

The present description also encompasses any kit comprising at least two of the herein described products, for example at least one or two distinct populations of particles, typically particles (B), for example a population of particles made of conductor particles and a population of particles made of insulator particles, preferably together with a removable device (C), and optionally together with a tool (such as one or more needles, one or more microneedles, a patch, an injector, etc.) designed to appropriately deposit and/or position the particles (B) at the adequate site of the subject's body.

The present description also encompasses a kit comprising herein described particles, typically particles (B), a herein described device, typically the removable device (C), and one or several tools selected from a sensor such as an electrode (for capturing neural data from the nervous system of a subject), a memory (for recording the captured neural data) and a processor (for processing the recorded neural data before sending the processed data back to the subject in the form of a signal perceivable by any one of the five natural senses of the subject). Preferably, in this kit, the removable device (C) is wearable by a subject, the size of particles is below 100 μm, and particles are i) prepared from a material selected from a conductor, a semiconductor, a semiconductor with direct bandgap, an insulator, a piezoelectric and a magnetoelectric material, and ii) activable by a signal emitted by the removable device (C).

Inventors herein describe a kit comprising at least two distinct populations of particles, optionally together with a tool designed to deposit and/or position particles at the adequate site of the subject's body for them to stably interact with hair follicles, biological cells of the dermis and/or epidermis, Low Threshold Mechanoreceptors (LTMRs) and/or end-organs of a subject, wherein the size of particles is below 100 μm, and particles are prepared from a material selected from a conductor, a semiconductor, a semiconductor with direct bandgap, an insulator, a piezoelectric and a magnetoelectric material, for example from a conductor, a semiconductor, a semiconductor with direct bandgap, a piezoelectric and a magnetoelectric material, preferably from a conductor, a semi-conductor, an insulator and a magnetoelectric material, even more preferably from a conductor, a semi-conductor and an insulator material. These particles are activable.

Also herein described is a kit comprising particles (as herein described), typically particles (B), a removable device (C) (as herein described), and one or several tools selected from a sensor such as an electrode, a memory and a processor, wherein the removable device (C) is wearable by a subject, the size of particles is below 100 μm, and particles i) are prepared from a material selected from a conductor, a semiconductor, a semiconductor with direct bandgap, an insulator, a piezoelectric and a magnetoelectric material, for example from a conductor, a semiconductor, a semiconductor with direct bandgap, a piezoelectric and a magnetoelectric material, preferably from a conductor, a semi-conductor, an insulator and a magnetoelectric material, even more preferably from a conductor, a semi-conductor and an insulator material, and ii) are activable by a signal emitted by the removable device (C).

### Applications

Inventors herein describe a system (typically the herein described “system (A)”) and its use for sensory enhancement in a subject, or for creating new sensory means in a subject, for example in a human being, allowing the subject to perceive in particular a physical signal, a chemical signal and/or a biological signal which are, or on the contrary which are not, perceived by the subject's senses, for example by a human sense.

They also herein describe particles (typically the herein described “particles (B)”) for use for touch sensory restoration in an amputee or in a burn victim, or for sensory substitution in a subject at least partially or totally deprived of taste, smell, hearing, balance and/or vision, when particles interact with hairs, hair follicles, biological cells of the dermis and/or epidermis, LTMRs and/or end-organs of the subject, preferably with hair follicles, biological cells of the dermis and/or epidermis, LTMRs and/or end-organs of the subject, and when particles are activated by an external source of energy. As herein above described, the size of the particles is preferably below 100 μm, and the particles are prepared from a material which may be selected from a conductor, a semiconductor, a semiconductor with direct bandgap, a piezoelectric and a magnetoelectric material and is preferably selected from a conductor, a semiconductor, a semiconductor with direct bandgap, an insulator, a piezoelectric and a magnetoelectric material, preferably from a conductor, a semiconductor, a semiconductor with direct bandgap, an insulator and a magnetoelectric material, or from a conductor, a semiconductor, a semiconductor with direct bandgap and an insulator material.

Sensory Touch Restoration

Only in the United States, about two (2) million persons are living with the loss of a limb. There is a high need for these subjects to recover the sense of touch. A natural sensory feedback through their prostheses is typically sought for these persons. The system of the present invention offers to the subjects exhibiting proper LTMRs and/or end-organs functioning a limb axon-like stimulation. The system has the advantage of being biocompatible and of remaining at the site of implantation. The system of the invention may be advantageously used to stimulate afferent sensory fibers and provide efficient sensory feedback.

Sensory Substitution

According to the WHO (World Health Organization), the number of people of all ages visually impaired was estimated to be 285 million in 2010, of whom 39 million were blind. Also, around 466 million people worldwide have disabling hearing loss, and 34 million of these are children. It is estimated that by 2050 over 900 million people will have disabling hearing loss. In view of these major global health issues, sensory substitution, typically through people's skin, could be a possibility for these people to “see” and/or “hear” with their skin.

Sensory Enhancement

The present invention now makes it possible to very significantly enhance, and even widen, the capacities of sensory perception offered to a subject, in particular to a human being, by its natural senses.

In the vision field, the present invention now allows a subject for example to beneficiate of a 360° vision, to see throughout the whole earth in real time (i.e., acquire remote vision), to see underwater, to acquire space vision and see for example activities and phenomena occurring at an atomic scale up to a visible scale in and outside our solar system to increase perception and understanding of the universe.

In the touch field, the present invention now allows a subject for example to perceive touch from another subject with whom he/she is not in physical contact with (remote touch sensation).

In the smell and/or taste fields, the present invention now allows a subject for example to perceive a noxious (odorless and tasteless by common sense) chemical or biological compound; or to perceive a biological change and typically be able to early diagnose a cancer or any other life-threatening disease from a biological, for example blood, sample of a subject for example with the sense of smell.

In the hearing field, the present invention now allows a subject for example to hear distant or remote (selected) sounds.

In the field of data (i.e., any information received as input signals which do not result from sensory experience or which cannot be perceived by a natural sense), the present invention now allows a subject for example to facilitate and/or increase data acquisition and/or processing (treatment) throughout daily activities, in particular in the context of learning.

New Sensory

Beyond sensory enhancement, new sensory could be acquired by a subject which would allow the subject to enlarge his/her perception of reality compared to the reality as perceived through his/her natural senses.

Non-limiting examples of new sensory perceptions include the access to vision outside the visible domain, such as for example in the U.V. and/or infrared domain; the access to sounds beyond current hearing ability such as for example the access to ultrasounds.

Typically, sensory restoration, sensory substitution, sensory enhancement, or the creation of new sensory means find application in a wide range of fields/industries/domains, such as in healthcare (typically by restoring senses and/or by substituting senses), in services (typically by enhancing life by providing assistance to persons), in communication, in defense/security (typically by making it possible to see, feel (touch), hear before it is accessible to normal human perception), in Aerospatiale (typically by augmenting knowledge), in agriculture, in automotive, in transports, in gaming, in sport, in entertainment (for example by augmenting entertainments experiences in music, in cinema), etc.

The embodiments of the invention described above are intended to be merely exemplary, and those skilled in the art will recognize, or will be able to ascertain using no more than routine experimentation, numerous equivalents of herein described specific materials, particles and compositions as well as equivalents of herein described methods or procedures. All such equivalents are considered to be within the scope of the invention and are encompassed by the appended claims.

### LEGENDS TO THE FIGURES

FIG. 1: Biological components of dermis and epidermis.

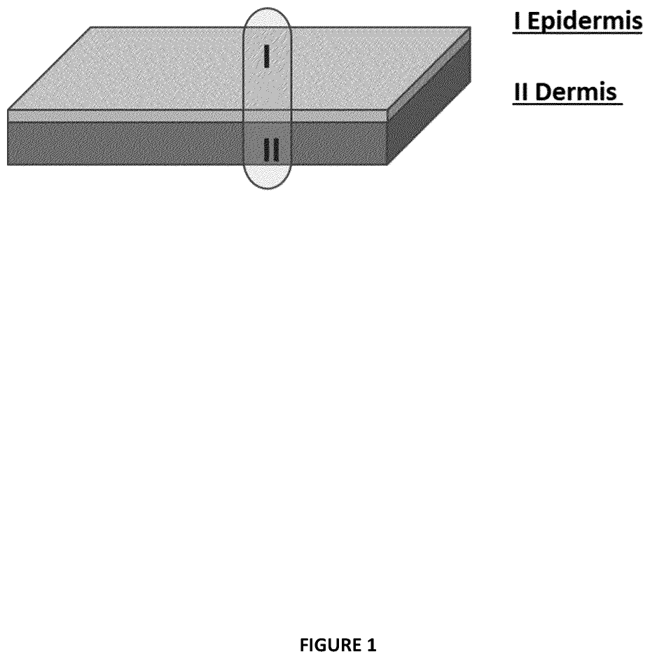

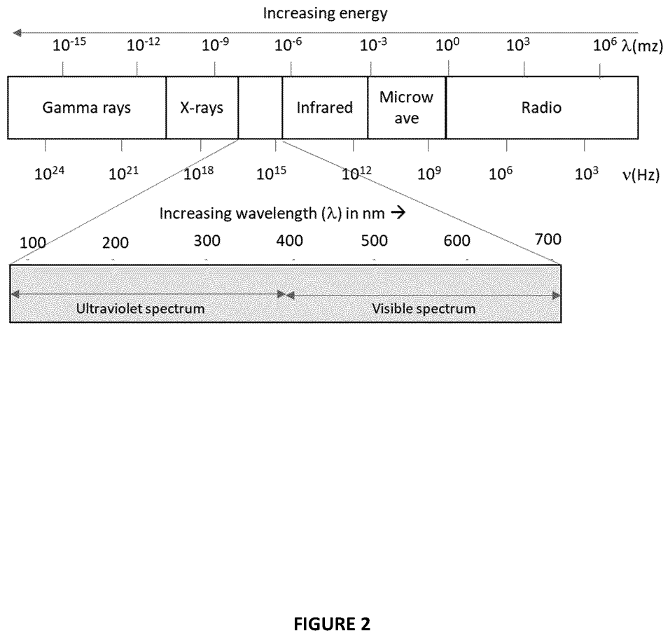

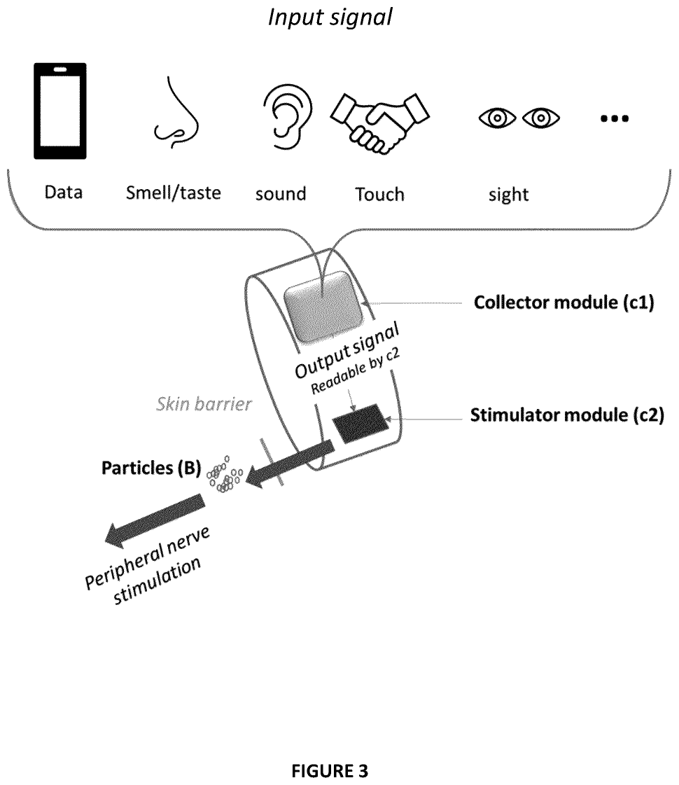

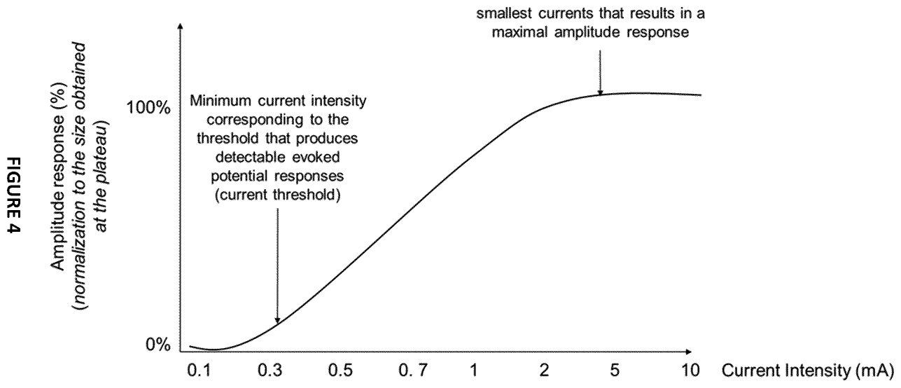

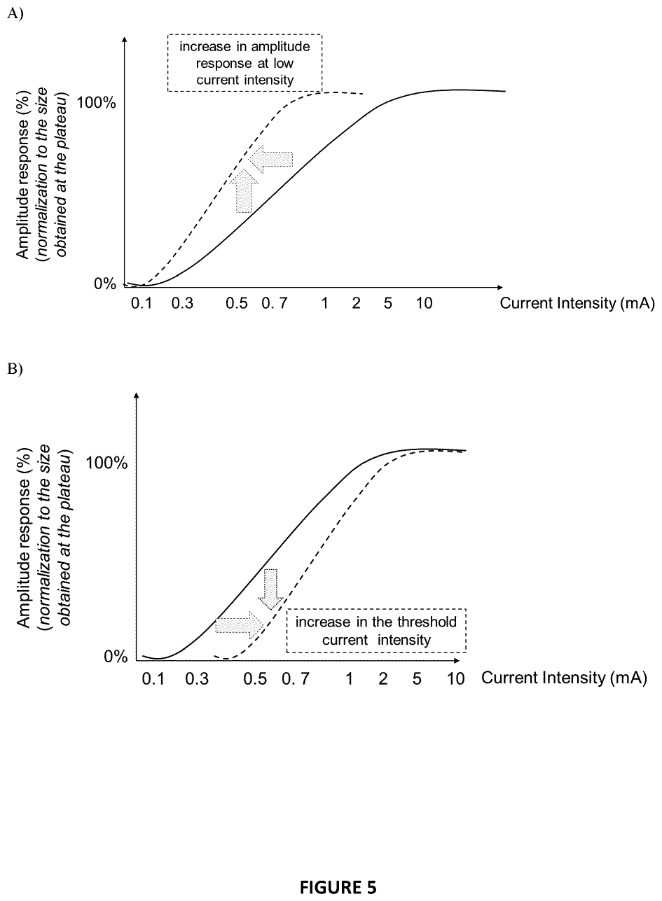

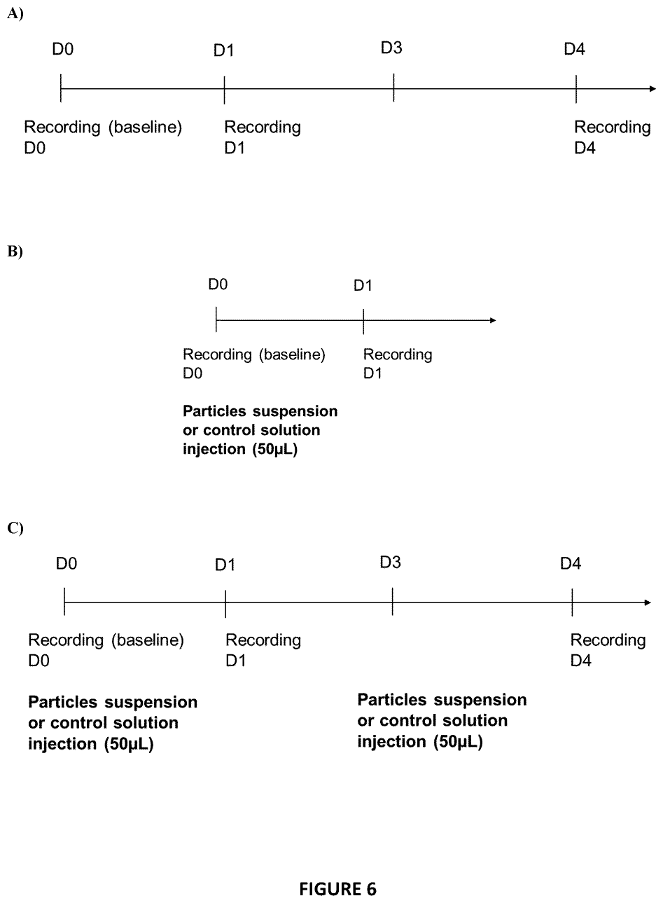

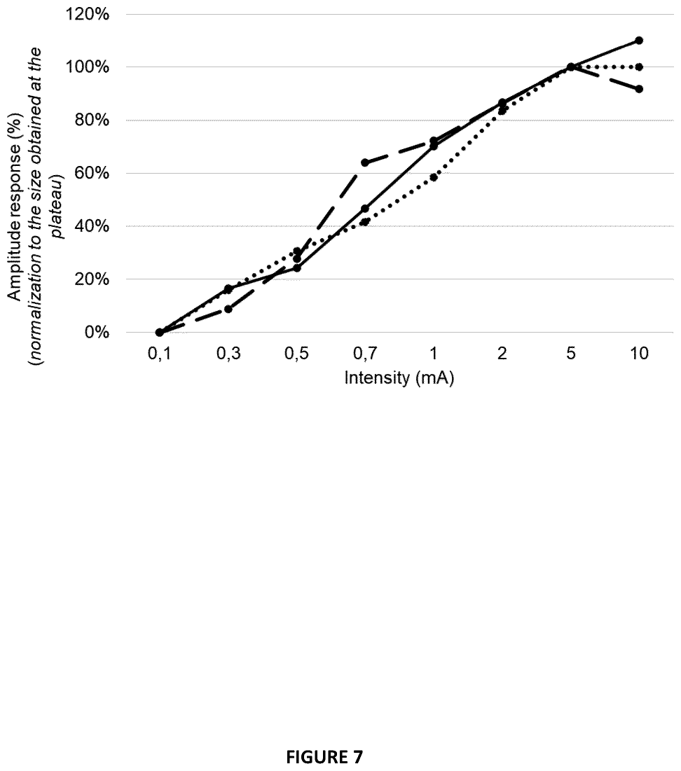

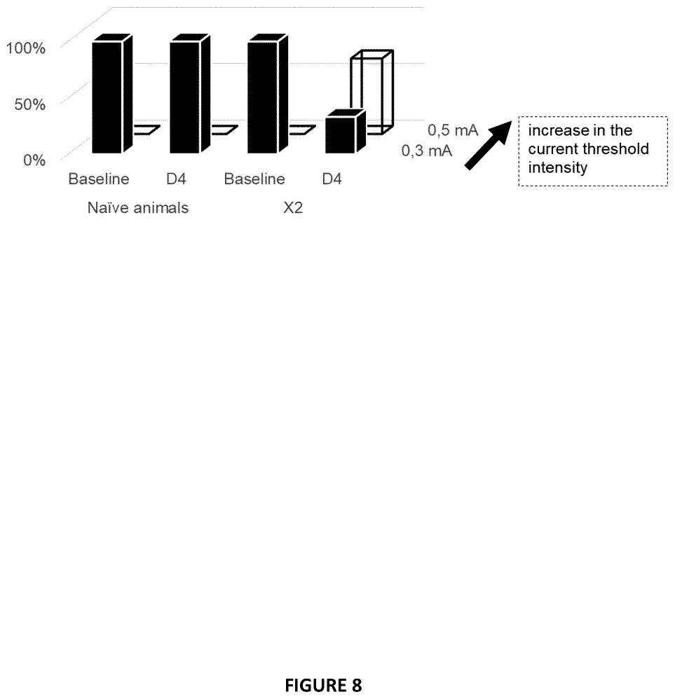

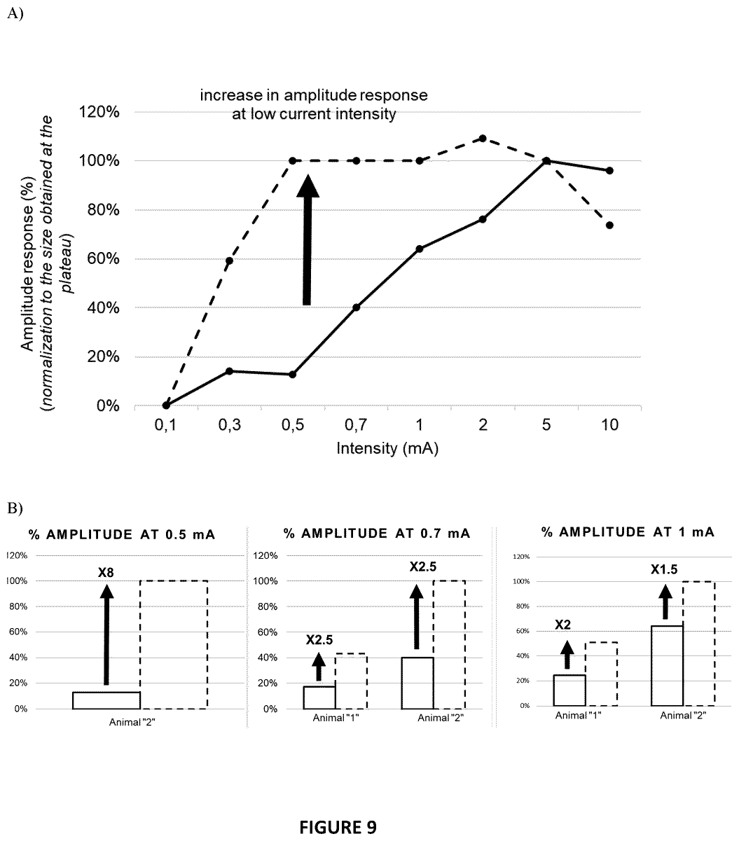

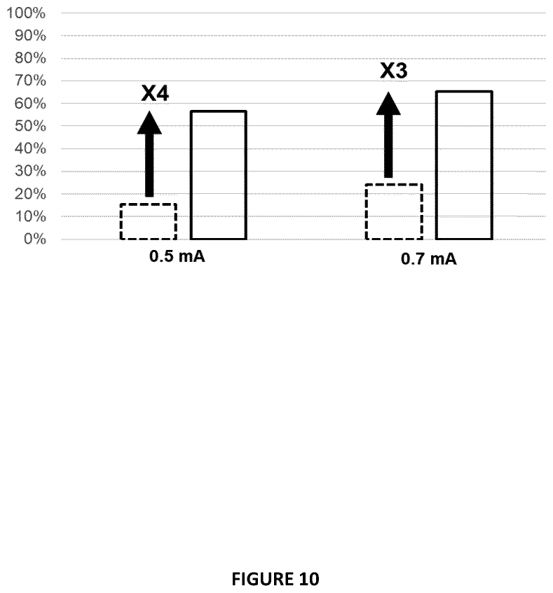

Epidermis (zone I). The epidermis comprises the stratum corneum (nonviable epidermis) layer, the stratum lucidum (viable epidermis) layer, the stratum granulosum (viable epidermis) layer, the stratum spinosum (viable epidermis) layer, and the stratum basal (viable epidermis) layer. The epidermis comprises the following biological cells: the keratinocytes which represent 95% of cells and are present in each layer, and the melanocytes, the Merkel cells, and the Langerhans cells which represent 5% of the remaining cells and are present in viable epidermis. The epidermis also comprises the following appendages: hairs (hairy skin), sweat glands, sebaceous glands and lipids.

Dermis (zone II). The dermis comprises the following biological cells: fibroblasts, mast cells, macrophages, lymphocytes and platelets. The dermis also comprises the following appendages: collagen fibrils, elastic connective tissue, mucopolysaccharides, highly vascularized network, lymph vessels, sensory nerves/nerve fibers, free nerve endings, end-organs such as Pacinian corpuscles, Meissner corpuscles, Ruffini corpuscles and/or longitudinal lanceolate endings, hair follicles, sebaceous gland and sweat glands.

FIG. 2: Electromagnetic signals from the electromagnetic spectrum showing the range of wavelengths and frequencies spanned by electromagnetic radiations.

FIG. 3: System (A) comprising particles (B) and a removable device (C).

The particles (B) are below 100 μm, are stably interacting with hairs, hair follicles, biological cells of the dermis and/or epidermis, Low Threshold Mechanoreceptors (LTMRs) and/or end-organs, preferably with hair follicles, biological cells of the dermis and/or epidermis, Low Threshold Mechanoreceptors (LTMRs) and/or end-organs, and are activated by a signal emitted by the removable device (C). The removable device (C) collects an input signal which is, optionally processed and, used to activate the particles (B), the removable device being wearable by a subject.

The device (C) typically comprises:
a collector module (c1) collecting an input signal which is selected from a physical signal, a chemical signal and/or a biological signal. The input signal may typically be a physical signal, a chemical signal and/or a biological signal perceived by our natural senses, or be a physical, chemical and/or biological signal which cannot be perceived by one of the five natural senses (such as an infrared signal, an ultrasound signal, etc.). The collector module may comprise a collector module (c1′) collecting an input signal and a processing module (c1″) encoding the input signal into an output signal readable by the stimulator module (c2);
a stimulator module (c2) comprising a source of energy which is selected from an electrical source, a light source, a magnetic source and a mechanical source, said source using the output signal to activate the particles (B).

The spikes, generated in response to input signal(s) from the collector module, confirm the successful reading of the output signal by the stimulator module present in the system (A) as well as the successful stimulation of the particles by the source of energy used to stimulate the peripheral nerves which will then convey/transmit a signal to the central nervous system which it can interpret.

FIG. 4: Schematic representation of a stimulus (current)/amplitude response curve.

Schematic representation of a theoretical stimulus (current)/amplitude response curve recorded in a Sensory Nerve Conduction (SNC) experiment. The amplitude response is given in % and normalized to the size of amplitude obtained at the plateau (i.e., the maximal amplitude response).

FIG. 5. Schematic representation of a stimulus (current)/amplitude response curve when conductor, semiconductor or insulator particles of the invention are present.

(A) Schematic representation of a theoretical stimulus/response curve when semiconductor or conductor particles (B) are stably interacting with hair follicles, biological cells of the dermis and/or epidermis, Low Threshold Mechanoreceptors (LTMRs) and/or end-organs, and are activated by the signal emitted by the removable device (C) (herein typically an electrical signal generated by a stimulating electrode). In dotted black line, the amplitude response curve in presence of conductor or semiconductor particles is shifted to the left when compared to the amplitude response curve in the absence of any particles (in full black line).

(B) Schematic representation of a theoretical stimulus/response curve when insulator particles (B) are stably interacting with hair follicles, biological cells of the dermis and/or epidermis, Low Threshold Mechanoreceptors (LTMRs) and/or end-organs, and are activated by the signal emitted by the removable device (C) (herein typically an electrical signal generated by a stimulating electrode). In dotted black line, the amplitude response curve in presence of insulator particles is shifted to the right when compared to the amplitude response curve in the absence of any particles (in full black line).

FIG. 6. Experimental procedure.

(A) Naïve animals; (B) Animals receiving one injection of “control solution” or “particles' suspensions” (X1) at day 0 (D0); (C) Animals receiving two injections of “control solution” or “particles' suspensions” (X2, X3) at day 0 (D0) and day 3 (D3).

FIG. 7. stimulus (current)/response curve of animal subcutaneously injected with “control solution”.

Current threshold (0.3 mA) and stimulus response curve observed in one animal subcutaneously injected with “control solution” at day 0 (D0) and day 3 (D3). Baseline recording (at D0) is represented in dotted black line, recording at day 1 (D1) is represented in full black line and recording at day 4 (D4) is represented in large dotted black line. The stimulus response curve is normalized to the size obtained at the plateau (which corresponds to a current intensity of 5 mA).

FIG. 8. Current threshold of animals subcutaneously injected with “particles' suspension” X2.

Current threshold observed at baseline (day 0, D0) and at day 4 (D4) for naïve animals (2 animals) and “particles' suspension” X2 (3 animals). At baseline (D0), 100% of animals in all groups presented a current threshold at 0.3 mA. At D4, 100% of animals from “naïve animal” group presented a current threshold at 0.3 mA whereas only 33% of animals from “particles' suspension X2” group presented a current threshold at 0.3 mA. Instead, at D4, 67% of animals from “particles' suspension X2” group presented a current threshold at 0.5 mA.

FIG. 9. Stimulus (current)/response curve of animals subcutaneously injected with “particles' suspension” X1.

(A) Amplitude response (at day 1, D1) observed in one rat subcutaneously injected with “particles' suspension” X1 (doted black line) when compared to baseline (day 0, D0) (full black line). A left shift of the curve due to an increase of amplitude response at low current intensity is observed in the rat with particles X1 when compared to baseline (no particles). (B) The percentage (%) of amplitude response at current intensity between 0.5 mA and 1 mA for rats (2 animals) with particles X1 (D1, dotted black line) is increased by more than 1.5 when compared to the % of amplitude response at baseline (D0, full black line).

FIG. 10. Stimulus (current)/response curve of animal subcutaneously injected with “particles' suspension” X3.

The percentage (%) of amplitude response (at day 4, D4) at current intensity 0.5 mA and 0.7 mA for one rat subcutaneously injected with particles X3 (full black line) at day 0 (D0) and day 3 (D3) is increased by more than 3 when compared to the % of amplitude response at baseline (D0), dotted black line).

### EXPERIMENTAL PART

### Particles of the Invention

Particles can be manufactured/synthesized according to synthesis methods described in the literature. Characterization of these “as synthesized particles” typically includes the analysis of particles size, composition and structure, the analysis of the composition and surface charge of the particles' surface, as well as the analysis of the hydrophilic or hydrophobic behavior of the particles.

Typical particles syntheses are described for example in the following publications:
Semiconductor particles of FeSe [J. Kwon et al. FeSe quantum dots for in vivo multiphoton biomedical imaging. Science Advances 2019; 5: eaay0044];
Conductor particles made of gold [G. Frens. Controlled Nucleation for the Regulation of the Particle Size in Monodisperse Gold Suspensions. Nature Physical Science volume 241, pages 20-22(1973)];
Conductor particles made of ReO₂ [A. L. Ivanovskii et al. Structure and electronic properties of new rutile-like rhenium (IV) dioxide ReO₂. Physics Letters A 348 (2005) 66-70];
Conductor particles made of Poly(3,4-ethylenedioxythiophene) [E. Cloutet et al. Synthesis of PEDOT latexes by dispersion polymerization in aqueous media. Materials Science and Engineering: C Volume 29, Issue 2, 1 Mar. 2009, Pages 377-382];
Insulator (piezoelectric or not piezoelectric) particles made of BN [A. Merlo et al. Boron nitride nanomaterials: biocompatibility and bio-applications. Biomater. Sci., 2018, 6, 2298].

### Protocol

In a typical experiment, the selected particles of the invention are administered on one, several or each of the following sites: hairs, hair follicles, biological cells of the dermis and/or epidermis, Low Threshold Mechanoreceptors (LTMRs) and end-organs, preferably hair follicles, biological cells of the dermis and/or epidermis, Low Threshold Mechanoreceptors (LTMRs) and end-organs. Particles are subsequently activated by an appropriate external source of energy.

The recording of a signal at the peripheral nervous system level or at the central nervous system level, confirms the activation of the particles and their action on the nervous system. Concretely, an output signal read by the stimulator module (c2) comprising the appropriate source of energy is converted into a signal that stimulates the peripheral nerves. Then, the peripheral nerves convey the information to the brain for neural coding and touch sensory restoration, sensory substitution, sensory enhancement or new sensory perception.

### Evaluation of the Effect of Particles of the Invention on Sensory Nerve Conduction (SNC) in the Caudal Nerves of Rats.

### Preamble

In the present experiment, the impact of subcutaneous administration of particles of the invention on the orthodromic sensory nerve conduction (SNC) was studied in the caudal nerve of rats. The investigation was conducted after 1 injection (on day 0, D0) or after 2 injections (on DO and day 3, D3) of the particles of the invention.

Sensory nerve action potential (SNAP) was obtained by stimulating sensory fibers and recording the nerve action potential (AP) at a point further along that nerve. The SNAP is a sum of APs of all stimulated nerve fibers in the tested nerve (in the present example, the caudal nerve of the rat). The SNAP onset indicates the AP arrival at the recording site (i.e., the recording electrode). The onset latency is also the time of the AP propagation between the stimulating and recording sites (i.e., the time to complete the distance between the stimulating and the recording electrodes), and can be used to compute the conduction velocity. In SNAP measurement, the onset latency depends on the fastest conducting nerve fibers and the conduction velocity reflects conduction in the fastest axons, while peak latency is an expression of the mean conduction velocity value among all nerve fibers participating in the SNAP. Recording the SNAP orthodromically refers to distal nerve stimulation and AP recording more proximally (the direction in which physiological sensory conduction occurs in the living subject).

The SNAP amplitude (typically expressed in μV) represents the number of sensory nerve fibers activated when exposed to a given current intensity (typically expressed in mA). When increasing the current intensity, a threshold current is first observed which corresponds to the minimal current intensity that produces detectable action potential responses. As the current intensity further increases, more sensory nerve fibers become activated and the SNAP amplitude increases. This will continue until all nerve fibers supplying the tested nerve are stimulated. The amplitude response therefore reaches a maximum value beyond which further increase of current intensity does not trigger further increase of activated sensory nerve fibers. Such intensity is called “maximal” (see a typical theoretical stimulus (current)/response curve on FIG. 4).

In the context of the present example, the particles of the invention (particles (B)) are intended to work through an “on”/“off” mode of action, meaning that when stably interacting with hair follicles, biological cells of the dermis and/or epidermis, Low Threshold Mechanoreceptors (LTMRs) and/or end-organs, and activated by an external source of energy, they act as transducers and convert the incoming signal into an output signal of different nature, or modulate/relay locally the incoming signal, thereby acting on peripheral nerves to convey an information to the brain for neural coding (i.e., processing of information).

In this context, FIG. 5A presents the theoretical stimulus (current)/response curve when semiconductor or conductor particles are used, and FIG. 5B presents the theoretical stimulus (current)/response curve when insulator particles are used, i.e., when they are inserted into the skin and are then activated by an external electrical source of energy (in the present example, by a stimulating electrode).

The semiconductor or conductor particles of the invention will typically create, where they are located/administered/injected, a “high conducting medium/spot”. Therefore, under a given current intensity stimulus, they will modulate/enhance locally the number of activated nerve fibers (i.e., increase the amplitude of the response and/or decrease the current threshold), when compared to the number of nerve fibers activated in the absence of any semiconductor or conductor particles (i.e., resulting in a left shift of the stimulus/response curve).

On the contrary, the insulator particles of the invention will typically create, where they are located/administered/injected, an “insulating medium/spot”. Therefore, under a given current intensity stimulus, they will modulate/decrease locally the number of activated nerve fibers (i.e., increase the current threshold and/or decrease the amplitude of the response), when compared to the number of nerve fibers activated in the absence of any insulator particles (i.e., resulting in a right shift of the stimulus/response curve).

However, for insulator, semiconductor and conductor particles, the maximum amplitude response value is not expected to be modified as the total volume/number of nerve fibers that can be activated when increasing the current intensity remains constant (corresponding to the total number of nerves fibers of the tested nerve).

### Materials and Methods

### Test Animals

Adult female Sprague-Dawley rats of about 6 and 12 weeks of age were used.

### Particles of the Invention

Particles X1, X2, X3 were supplied as suspension (“particles' suspensions”) in sterile tubes.

X1 corresponds to particles made of gold.

X2 corresponds to particles made of boron nitride.

X3 corresponds to particles made of graphene (i.e., particles made of carbon atoms).

All “particles' suspensions” for injection were prepared at room temperature within typically 4 hours prior subcutaneous injection as follows:

a) each tube containing the particles suspension was prepared by adding a sterile solution of glucose in order to have a suspension ready for injection (i.e., with the appropriate osmolarity for animal subcutaneous injection). A “control solution” was prepared by diluting sterile solution of glucose in water for injection to a final concentration in glucose equal to 5%; and

b) the as prepared “particles' suspensions” and “control solution” were vortexed for 5 minutes, and

c) the “particles' suspension” and “control solution” were used within 4 hours.

### Experimental Procedure

Rats were randomly distributed in experimental groups with 3 or 4 rats per group. Two (2) naïve rats served as control without any injection (See FIG. 6 for the schematic representation of the experimental procedure).

### Step 1: Baseline Recording

At day 0 (D0), all rats were anaesthetized using isoflurane-oxygen using a nose cone. The orthodromic SNAP recording was performed with an electromyograph. Subcutaneous monopolar needle electrodes were used for both stimulation and recording at the animal's tail. However, for the experiments, the stimulating electrodes were not implanted in the animals' tails but remained in contact with the surface of the animals' tails (i.e., they were used as “transcutaneous” electrodes, meaning that only the current penetrates the skin). The stimulating and recording electrode anodes were separated by a fixed standard distance (50 mm) with the recording electrode close to the tail base. A minimum distance between the recording electrode and the stimulating electrode located above the biological area where the particles are located is required (preferably at least 2 cm, for example 5 cm as in the present experiment). A ground was placed between the stimulating and recording electrodes.

SNAP recording was performed at incremented stimulus intensity (typically from 0.1 mA to 10 mA, such as: 0.1 mA-0.3 mA-0.5 mA-0.7 mA-1 mA-2 mA-5 mA-10 mA). Each stimulation pulse was a monophasic square wave current of 200 μs duration. The caudal nerve was stimulated with 20 series of pulses at a frequency of 1 Hz and the arithmetic average of the SNAP signal was recorded.

Typical SNAP parameters analyzed were:
the minimal current intensity corresponding to the threshold that produces detectable (evoked) action potential responses (current threshold);
the amplitudes of SNAP and the smallest currents that result in a maximal amplitude response (stimulus (current)/response curve);
the “onset latency”, “peak latency” and sensory nerve conduction velocity.

### Step 2: Impact of Particles of the Invention on Snap

Under animal anesthesia, “control solution” or “particles' suspensions” (X1, X2, X3) were subcutaneously administered to the animal (day 0, D0) at a volume of 50 μL. The site of injection was located just under the stimulating electrode.

Twenty-four (24) hours following “control solution” or “particles' suspensions” administration (Day 1, D1), SNAP recording as described in STEP 1 was repeated.

Specifically, for “particles' suspensions” X2 and X3, and “control solution”, 72 hours after the first injection (i.e., at day 3, D3) a second subcutaneous administration was performed at the same site of injection. The site of injection was located just under the stimulating electrode. Twenty-four (24) hours following the second administration (days 4, D4), SNAP recording as described in STEP 1 was repeated.

### Results

### Clinical Sign and Body Weight

There was no macroscopically visible change in the behavior of rats after the injection of the “particles' suspensions” or of the “control solution”. There was no sign of body weight loss during the study.

### SNAP Measures.

Animals Subcutaneously Injected with “Control Solution”

FIG. 7 represents a typical stimulus response curve obtained for animals subcutaneously injected with the “control solution”. The current threshold is 0.3 mA and the minimum current intensity that results in a maximal amplitude response is observed at 5 mA. A repeatable stimulus response curve is observed at 3 different time points, i.e., DO (baseline recording), D1 and D4.

“Control solution” and naïve animals showed similar results (data not shown), highlighting the absence of impact of vehicle (sterile glucose 5%) injection on SNAP measures.

For all “particles' suspensions” the maximal amplitude response fell between the maximal amplitude response found in both naïve animals and animal injected with the “control solution”. The minimum current intensity that produced the maximum response was about 5 mA. Therefore, normalization of the amplitude response to the size obtained at 5 mA was used to interpret the stimulus/response curves of “particles' suspensions” groups.

### “Particles Suspensions” Groups

### “Particles' Suspension” X2

At D0, D1 and D4, a current threshold of about 0.3 mA was observed for naïve rats and “control solution”. However, an increase in the threshold intensity (from 0.3 mA to 0.5 mA) was observed in 1 out of 3 rats at D1 and in 2 out of 3 rats at D4 for “particles' suspension” X2, indicating a right shift of the stimulus response curve. The increased number of rats presenting a threshold intensity at 0.5 mA was correlated with the increased number of particles X2 subcutaneously injected (FIG. 8).

### “Particles' Suspension” X1

Rats with subcutaneous injection at DO of “particles' suspension” X1 beneath the stimulating electrode showed at D1 a left shift of the stimulus/response curves with typically a more than 1.5-fold increase of the percentage of amplitude response observed at current intensity between 0.5 mA and 1 mA when compared to baseline (no particles) (FIG. 9).

### “Particles' Suspension” X3

Rats with subcutaneous injection at DO and D3 of “particles' suspension” X3 showed at D4 a more than 3-fold increase of the percentage of amplitude response observed at current intensity 0.5 mA and 0.7 mA when compared to baseline (no particles), corresponding to a left shift of the stimulus/response curves when compared to baseline (no particles) (FIG. 10).

### Onset Latency, Peak Latency and Conduction Velocity (Data not Shown)

The onset response latency reflects the action potential propagation time for the largest, fastest sensory axons and was used to calculate the sensory nerve conduction velocity (SNCV). SNCV was already found maximal at the current stimulus threshold. There was no major difference in terms of onset latency and SNCV among all groups.

The peak latency reflects the latency (conduction velocity) along the majority of axons and is measured at the peak of the action potential. There was no major difference in the peak latency among all groups.

### CONCLUSION

The obtained results indicate that:
the treatment with “control solution” did not modify the SNAP response as measured in the caudal nerves of rats when compared to naïve animals.
X2 particles of the invention shifted the stimulus/response curves (amplitude) to the right, which showed a reduced number of fibers that responded to the given intensity (below 5 mA) when compared to animals without particles.
X1 and X3 particles of the invention did not interfere with the SNAP and shifted the stimulus/response curves (amplitude) to the left, which showed an increased number of fibers that responded to the given intensity (below 5 mA) when compared to animals without particles.
None of the tested particles affected the maximal stimulation (current) intensity, showing a lack of interference with the excitability of the majority of excitable fibers at high stimulation intensity.

The results showed the efficiency of the herein described particles of the invention to modulate incoming external signal(s), thanks to the stable interaction of the particles with hair follicles, biological cells of the dermis and/or epidermis, Low Threshold Mechanoreceptors (LTMRs) and/or end-organs (stability being directly correlated to the design of the particles), and their activation by the signal emitted by the removable device (C) (herein the electrical signal generated by the stimulating electrode), thereby acting on peripheral nerves to convey an information to the brain for neural coding.

Interestingly, multiple injections with conductor, semiconductor and/or insulator “particles suspensions” can be performed thereby offering the possibility of creating multiple levels of electrical modulation of the nerve fibers for coding.

Alternatively, other external source of energy can be used. Typically, for particles made of semiconductors with direct band gap material or for particles made of conductors prepared with carbon atoms, such as graphene, a light source can be used to activate the particles which will thus be capable of acting on peripheral nerves to convey an information to the brain for neural coding.

A similar system can be used also to restore or enhance the functioning of organ(s) or tissue(s) by allowing the stimulation of motor nerve(s).

1-19. (canceled)

20. A system (A) comprising particles (B) and a removable device (C), wherein
the size of particles (B) is below 100 μm, particles (B) are stably interacting with hair follicles, biological cells of the dermis and/or epidermis, Low Threshold Mechanoreceptors (LTMRs) and/or end-organs of a subject, and particles (B) are prepared from a material selected from a conductor, a semiconductor, a semiconductor with direct bandgap, an insulator, a piezoelectric and a magnetoelectric material, and are activable by a signal emitted by the removable device (C), and
the removable device (C) collects an input signal which is, optionally processed and, used to activate the particles (B), the removable device being wearable by a subject.

21. The system according to claim 20, wherein the biological cell is selected from a keratinocyte, melanocyte, Merkel cell, Langerhans cell, fibroblast, mast cell, macrophage, lymphocyte and platelet, the LTMR is selected from SAI-LTMR, SAII-LTMR, RAI-LTMR, RAII-LTMR, Aδ-LTMR and C-LTMR and/or the end-organ is selected from Ruffini corpuscle, Meissner corpuscle, Pacinian corpuscle and longitudinal lanceolate ending.

22. The system according to claim 20, wherein the removable device (C) is stably interacting with particles and, both the removable device and particles are not located at a biological area of the subject corresponding to fingertips, mouth, lips and foot soles.

23. The system according to claim 20, wherein the device (C) comprises a collector module (c1) collecting an input signal which is selected from a physical signal, a chemical signal and a biological signal, the collector module (c1) being capable of processing the signal when required, and a stimulator module (c2).

24. The system according to claim 23, wherein the collector module (c1) comprises a module (c1′) collecting an input signal and a processing module (c1″) encoding the input signal into an output signal readable by the stimulator module (c2).

25. The system according to claim 23, wherein the stimulator module (c2) comprises a source of energy which is selected from an electrical source, a light source, a magnetic source and a mechanical source, said source using the output signal to activate the particles (B).

26. The system according to claim 23, wherein the device (C) is included in jewelry, in clothing or in a medical device.

27. The system according to claim 26, wherein the device (C) is a bracelet, a ring, a necklace, an artificial skin, a patch, a bandage, a mitt or a glove.

28. The system according to claim 20, wherein particles (B) are incorporated in a composition which is a liquid, a tattoo ink, or a gel, or are part of a needle or a microneedle or are a part of a tip of a needle or microneedle.

29. The system according to claim 25, wherein:
i) when the source of energy is an electrical source, the particle is prepared from a material selected from a conductor, a semi-conductor, an insulator and a piezoelectric material;
ii) when the source of energy is a light source, the particle is prepared from a material selected from a semiconductor with a direct band gap material and a conductor made of carbon atoms;
iii) when the source of energy is a mechanical source, the particle is prepared from a piezoelectric material; and
iv) when the source of energy is a magnetic source, the particle is prepared from a magnetoelectric material.

30. A method for sensory enhancement in a healthy subject or for creating new sensory means in a healthy subject comprising providing said healthy subject with a system according to claim 20 and implanting or injecting particles (B) into a site of said subject to allow the perception of a physical signal, chemical signal and/or biological signal which are not perceived by a sense of the subject when stimulated by the removable device (C).

31. A method for providing touch sensory restoration in an amputee or in a burn victim, or for sensory substitution in a subject at least partially deprived of taste, smell, hearing, balance and/or vision comprising implanting or injecting particles into said amputee, burn victim, or subject at least partially deprived of taste, smell, hearing, balance and/or vision or a composition comprising said particles, such that said particles interact with hair follicles, biological cells of the dermis and/or epidermis, LTMRs and/or end-organs of the subject and activating said particles or said composition comprising said particles with an external source of energy, wherein the size of each particle is below 100 μm, and the particles are prepared from a material selected from a conductor, a semiconductor, a semiconductor with direct bandgap, an insulator, a piezoelectric and a magnetoelectric material.

32. The method according to claim 31, wherein the composition is a liquid, a tattoo ink, or a gel.

33. The method according to claim 31, wherein the source of energy is selected from an electrical source, a light source, a mechanical source and a magnetic source.

34. The method according to claim 33, wherein:
i) when the source of energy is an electrical source, the particle is prepared from a material selected from a conductor, a semi-conductor, an insulator and a piezoelectric material;
ii) when the source of energy is a light source, the particle is prepared from a material selected from a semiconductor with a direct band gap material and a conductor made of carbon atoms;
iii) when the source of energy is a mechanical source, the particle is prepared from a piezoelectric material; and
iv) when the source of energy is a magnetic source, the particle is prepared from a magnetoelectric material.

35. A kit comprising at least two distinct populations of particles, optionally together with a tool designed to deposit and/or position particles at the adequate site of the subject's body for them to stably interact with hair follicles, biological cells of the dermis and/or epidermis, Low Threshold Mechanoreceptors (LTMRs) and/or end-organs of a subject, wherein the size of particles is below 100 μm, and particles are prepared from a material selected from a conductor, a semiconductor, a semiconductor with direct bandgap, an insulator, a piezoelectric and a magnetoelectric material, and are activable.

36. The kit according to claim 35, wherein the tool is one or more needles, one or more microneedles, a patch or an injector.

37. A kit comprising particles (B), a removable device (C), and one or several tools selected from a sensor, an electrode, a memory and a processor, wherein the removable device (C) is wearable by a subject, the size of particles (B) is below 100 μm, and particles (B) are i) prepared from a material selected from a conductor, a semiconductor, a semiconductor with direct bandgap, an insulator, a piezoelectric and a magnetoelectric material, and ii) activable by a signal emitted by the removable device (C).
